SEQUENCE LISTING (7 sequences)

SEQ ID NO: 1 - protein, 943 aa, Artificial Sequence
  Description of Artificial Sequence Synthetic polypeptide
GGYGPGAGQQGPGSGGQQGPGGQGPYGSGQQGPGGAGQQGPGGQGPYGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAA

SEQ ID NO: 2 - protein, 315 aa, Artificial Sequence
  Description of Artificial Sequence Synthetic polypeptide
GGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAA

SEQ ID NO: 3 - protein, 90 aa, Artificial Sequence
  Description of Artificial Sequence Synthetic polypeptide
  MISC_FEATURE (4)..(67): This region may encompass 4-8 repeating “GPG-X1” repeating units, wherein X1 is “SGGQQ,” “GAGQQ,” “GQGPY,” “AGQQ” or “SQ”
  MISC_FEATURE (7)..(11): This region may encompass “SGGQQ,” “GAGQQ,” “GQGPY,” “AGQQ” or “SQ”
  MISC_FEATURE (15)..(19): This region may encompass “SGGQQ,” “GAGQQ,” “GQGPY,” “AGQQ” or “SQ”
  MISC_FEATURE (23)..(27): This region may encompass “SGGQQ,” “GAGQQ,” “GQGPY,” “AGQQ” or “SQ”
  MISC_FEATURE (31)..(35): This region may encompass “SGGQQ,” “GAGQQ,” “GQGPY,” “AGQQ” or “SQ”
  MISC_FEATURE (39)..(43): This region may encompass “SGGQQ,” “GAGQQ,” “GQGPY,” “AGQQ” or “SQ”
  MISC_FEATURE (47)..(51): This region may encompass “SGGQQ,” “GAGQQ,” “GQGPY,” “AGQQ” or “SQ”
  MISC_FEATURE (55)..(59): This region may encompass “SGGQQ,” “GAGQQ,” “GQGPY,” “AGQQ” or “SQ”
  MISC_FEATURE (63)..(67): This region may encompass “SGGQQ,” “GAGQQ,” “GQGPY,” “AGQQ” or “SQ”
  MISC_FEATURE (71)..(90): This region may encompass 6-20 residues
GGYGPGXXXXXGPGXXXXXGPGXXXXXGPGXXXXXGPGXXXXXGPGXXXXXGPGXXXXXGPGXXXXXGPSAAAAAAAAAAAAAAAAAAAA

SEQ ID NO: 4 - protein, 5 aa, Artificial Sequence
  Description of Artificial Sequence Synthetic peptide
SGGQQ

SEQ ID NO: 5 - protein, 5 aa, Artificial Sequence
  Description of Artificial Sequence Synthetic peptide
GAGQQ

SEQ ID NO: 6 - protein, 5 aa, Artificial Sequence
  Description of Artificial Sequence Synthetic peptide
GQGPY

SEQ ID NO: 7 - protein, 4 aa, Artificial Sequence
  Description of Artificial Sequence Synthetic peptide
AGQQ

(19) United States
(12) Patent Application Publication
(10) Pub. No.: US 2019/0389916 A1
(43) Pub. Date: Dec. 26, 2019

(54) Molded Bodies Comprising Blends of Cellulose and Recombinant Silk Polypeptides

(71) Applicant: Bolt Threads, Inc., Emeryville, CA (US)

(72) Inventors: Gregory Wilson Rice, South San Francisco, CA (US); Steven Tom, Berkeley, CA (US); Hua Li, Davis, CA (US); Amir Ahmad Bakhtiary Davijani, Oakland, CA (US); Jessica Wang, Oakland, CA (US); Lindsay Wray, Benicia, CA (US); Sandra Lorena Castano Garcia, Hayward, CA (US); Zoya Nasir Zulfiqar, Oakland, CA (US); Jeroen Visjager, Emeryville, CA (US)

(21) Appl. No.: 16/409,480

(22) Filed: May 10, 2019

Related U.S. Application Data
(60) Provisional application No. 62/669,904, filed on May 10, 2018.

(51) Int. Cl. C07K 14/435; D02J 11/00; D01F 2/24; D01F 11/02

(52) U.S. Cl. CPC C07K 14/43518; D02J 11/00; D10B 2211/04; D01F 11/02; D10B 2201/20; D01F 2/24

(57) ABSTRACT

Provided herein are scalable methods of spinning fiber comprising bi-constituent blends of cellulose and recombinant spider silk polypeptides. Also provided are compositions comprising blended fibers of cellulose and recombinant spider silk polypeptides.

### REFERENCE TO RELATED APPLICATIONS

This application claims the benefit of U.S. Provisional Application 62/669,904, filed May 10, 2018, which is hereby incorporated by reference in its entirety, for all purposes.

### SEQUENCE LISTING

The instant application contains a Sequence Listing which has been submitted via EFS-Web and is hereby incorporated herein by reference in its entirety. Said ASCII copy, created on Month XX, 20XX, is named XXXXXUS_sequencelisting.txt, and is X,XXX,XXX bytes in size.

### BACKGROUND

Biorenewable and biodegradable materials are of increasing interest as an alternative to petroleum-based products. To this end, considerable effort has been made to develop methods of making materials and fibers from molecules derived from plants and animals. Fiber made from regenerated protein dates back to the 1890s and has been made using various traditional wet-spinning techniques. Cellulose has been wet-spun and dry-wet spun into fiber since the 1850s.

Silk proteins such as silk fibroin and spidroins have a complex secondary structure which make them an ideal candidate for the creation of protein-based materials such as fiber. Similarly, cellulose is a widely-available inexpensive feedstock that forms crystalline structures and has been widely used in the apparel industry.

Due to these similarities, there has been interest in generating fibers comprising both cellulose and regenerated silk fibroin. However, the properties of fibers comprising cellulose and recombinant spider silk proteins have not been investigated. As such, unique properties that are not found in either fiber are unknown.

The present disclosure relates to scalable methods for producing molded bodies such as fibers comprising recombinant spider silk polypeptides blended with cellulosic components and the fibers produced by such methods.

### SUMMARY

Provided herein are fibers comprising recombinant spider silk polypeptide powder and cellulose, wherein the fiber has a percentage by weight of cellulose that ranges from 25%-75% and a percentage by weight of recombinant spider silk polypeptide powder that ranges from 75%-25% and wherein the fiber has a tenacity of at least 10 cN/tex. In one embodiment, the fiber has a tenacity of at least 20 cN/tex. In one embodiment, the fiber has a tenacity of at least 30 cN/tex. In one embodiment, the fiber has a tenacity of at least 40 cN/tex.

In some embodiments, the tenacity is measured in accordance with ATSM D3822-14.

In some embodiments, the fiber has a percentage by weight of cellulose ranging from 50%-90% and a percentage by weight of recombinant spider silk polypeptide powder ranging from 10%-50%. In other embodiments, the fiber has a percentage by weight of cellulose ranging from 75%-90% and a percentage by weight of recombinant spider silk polypeptide powder ranging from 10%-25%. In some embodiments, the fiber has a percentage by weight of cellulose ranging from 25%-90% and a percentage by weight of recombinant spider silk polypeptide powder ranging from 75%-10%.

In one embodiment, the fiber shrinks less than 5% by length when treated with water. In another embodiment, the fiber shrinks less than 4% by length when treated with water. In another embodiment, the fiber shrinks less than 3% by length when treated with water.

In some embodiments, the fiber is formed from dissolving the blend of cellulose and recombinant spider silk polypeptide powder in a solution comprising NMMO monohydrate and extruding a fiber. In one embodiment, extruding the fiber comprises extruding the fiber into a water bath. In another embodiment, the fiber is treated by annealing the fiber with an alcohol.

In some embodiments, the fiber has an improved luster after the fiber is treated by annealing the fiber with the alcohol.

In some embodiments, the recombinant silk polypeptide powder comprises a recombinant silk protein corresponding to SEQ ID NO: 1.

In some embodiments, the recombinant silk polypeptide powder has a purity of greater than 50%.

In some embodiments, the cellulose is microcrystalline cellulose. In some embodiments, the cellulose is alpha cellulose. In some embodiments, the cellulose is dissolving cellulose. In some embodiments, the fiber has a percentage elongation at break of at least 5%. In some embodiments, the fiber has a percentage elongation at break of at least 6%. In some embodiments, the fiber has a percentage elongation at break of at least 7%.

In some embodiments, the percentage elongation at break is measured in accordance with ATSM D3822-14. In some embodiments, the fiber forms mechanical interactions with itself.

In another aspect, provided herein are a staple yarn comprising a series of staple fibers formed from any of the fibers of the embodiments.

In another aspect, provided herein are a filament yarn comprising one or more of the fibers of the embodiments twisted around a common axis.

In another aspect, provided herein are methods of producing a fiber comprising a blend of cellulose and recombinant spider silk polypeptide powder, the steps comprising dissolving cellulose and a recombinant spider silk polypeptide powder in a solvent to form a solution and extruding the solution to create a fiber, wherein the fiber has a percentage by weight of cellulose ranging from 25%-90% and a percentage by weight of recombinant spider silk polypeptide powder ranging from 10%-75% and wherein the fiber has a tenacity of at least 10 cN/tex.

In one embodiment, the solvent is NMMO monohydrate. In one embodiment, the solution further comprises propyl gallate.

In some embodiments, extruding the fiber comprises extruding the fiber into a water bath. In another embodiment, extruding the fiber comprises extruding the fiber into a water bath comprising an alcohol.

In some embodiments, the steps further comprise annealing the fiber with an alcohol.

In one embodiment, the fiber has a tenacity of at least 20 cN/tex. In another embodiment, the fiber has a tenacity of at least 30 cN/tex. In another embodiment, the fiber has a tenacity of at least 40 cN/tex.

In one embodiment, the fiber has a percentage by weight of cellulose ranging from 50%-90% and a percentage by weight of recombinant spider silk polypeptide powder ranging from 10%-50%. In another embodiment, the fiber has a percentage by weight of cellulose ranging from 75%-90% and a percentage by weight of recombinant spider silk polypeptide powder ranging from 10%-25%. In another embodiment, the fiber has a percentage by weight of cellulose ranging from 25%-90% and a percentage by weight of recombinant spider silk polypeptide powder ranging from 75%-10%.

In some embodiments, the method further comprises extruding the fiber at an extrusion rate and winding the fiber on a first take-up device such that the jet stretch of the fiber is at least 7. In some embodiments, the method further comprises extruding the fiber at an extrusion rate and winding the fiber on a first take-up device such that the jet stretch of the fiber is at least 8. In some embodiments, the method further comprises extruding the fiber at an extrusion rate and winding the fiber on a first take-up device such that the jet stretch of the fiber is at least 9.

In some embodiments, the fiber is transferred to one or more consecutive water baths. In some embodiments, the fiber is not subject to any additional drawing as it is transferred between consecutive water baths.

### BRIEF DESCRIPTION OF THE DRAWINGS

The foregoing and other objects, features and advantages will be apparent from the following description of particular embodiments disclosed herein, as illustrated in the accompanying drawings in which like reference characters refer to the same parts throughout the different views. The drawings are not necessarily to scale, emphasis instead placed upon illustrating the principles of various embodiments disclosed herein.

FIG. 1 provides a graph of the percent shrinkage of fibers comprising different ratios of cellulose and recombinant spider silk powder (“18B Powder”) after treatment with water.

### DETAILED DESCRIPTION

The details of various embodiments disclosed herein are set forth in the description below. Other features, objects, and advantages will be apparent from the description. Unless otherwise defined herein, scientific and technical terms used in connection with the present disclosure shall have the meanings that are commonly understood by those of ordinary skill in the art. Further, unless otherwise required by context, singular terms shall include the plural and plural terms shall include the singular. The terms “a” and “an” includes plural references unless the context dictates otherwise. Generally, nomenclatures used in connection with, and techniques of, biochemistry, enzymology, molecular and cellular biology, microbiology, genetics and protein and nucleic acid chemistry and hybridization described herein are those well-known and commonly used in the art.

The following terms, unless otherwise indicated, shall be understood to have the following meanings:

The term “polynucleotide” or “nucleic acid molecule” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation.

Unless otherwise indicated, and as an example for all sequences described herein under the general format “SEQ ID NO:”, “nucleic acid comprising SEQ ID NO:1” refers to a nucleic acid, at least a portion of which has either (i) the sequence of SEQ ID NO:1, or (ii) a sequence complementary to SEQ ID NO:1. The choice between the two is dictated by the context. For instance, if the nucleic acid is used as a probe, the choice between the two is dictated by the requirement that the probe be complementary to the desired target.

An “isolated” RNA, DNA or a mixed polymer is one which is substantially separated from other cellular components that naturally accompany the native polynucleotide in its natural host cell, e.g., ribosomes, polymerases and genomic sequences with which it is naturally associated.

An “isolated” organic molecule (e.g., a silk protein) is one which is substantially separated from the cellular components (membrane lipids, chromosomes, proteins) of the host cell from which it originated, or from the medium in which the host cell was cultured. The term does not require that the biomolecule has been separated from all other chemicals, although certain isolated biomolecules may be purified to near homogeneity.

The term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

An endogenous nucleic acid sequence in the genome of an organism (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “recombinant” because it is separated from at least some of the sequences that naturally flank it.

A nucleic acid is also considered “recombinant” if it contains any modifications that do not naturally occur to the corresponding nucleic acid in a genome. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome.

The term “peptide” as used herein refers to a short polypeptide, e.g., one that is typically less than about 50 amino acids long and more typically less than about 30 amino acids long. The term as used herein encompasses analogs and mimetics that mimic structural and thus biological function.

The term “polypeptide” encompasses both naturally-occurring and non-naturally-occurring proteins, and fragments, mutants, derivatives and analogs thereof. A polypeptide may be monomeric or polymeric. Further, a polypeptide may comprise a number of different domains each of which has one or more distinct activities.

The term “isolated protein” or “isolated polypeptide” is a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art. As thus defined, “isolated” does not necessarily require that the protein, polypeptide, peptide or oligopeptide so described has been physically removed from its native environment.

The term “polypeptide fragment” refers to a polypeptide that has a deletion, e.g., an amino-terminal and/or carboxy-terminal deletion compared to a full-length polypeptide. In a preferred embodiment, the polypeptide fragment is a contiguous sequence in which the amino acid sequence of the fragment is identical to the corresponding positions in the naturally-occurring sequence. Fragments typically are at least 5, 6, 7, 8, 9 or 10 amino acids long, preferably at least 12, 14, 16 or 18 amino acids long, more preferably at least 20 amino acids long, more preferably at least 25, 30, 35, 40 or 45, amino acids, even more preferably at least 50 or 60 amino acids long, and even more preferably at least 70 amino acids long.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences.) As used herein, homology between two regions of amino acid sequence (especially with respect to predicted structural similarities) is interpreted as implying similarity in function.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art. See, e.g., Pearson, 1994, Methods Mol. Biol. 24:307-31 and 25:365-89 (herein incorporated by reference).

The twenty conventional amino acids and their abbreviations follow conventional usage. See Immunology-A Synthesis (Golub and Gren eds., Sinauer Associates, Sunderland, Mass., 2^{nd} ed. 1991), which is incorporated herein by reference. Stereoisomers (e.g., D-amino acids) of the twenty conventional amino acids, unnatural amino acids such as α-, α-disubstituted amino acids, N-alkyl amino acids, and other unconventional amino acids may also be suitable components for polypeptides. Examples of unconventional amino acids include: 4-hydroxyproline, γ-carboxyglutamate, ϵ-N,N,N-trimethyllysine, ϵ-N-acetyllysine, O-phosphoserine, N-acetylserine, N-formylmethionine, 3-methylhistidine, 5-hydroxylysine, N-methylarginine, and other similar amino acids and imino acids (e.g., 4-hydroxyproline). In the polypeptide notation used herein, the left-hand end corresponds to the amino terminal end and the right-hand end corresponds to the carboxy-terminal end, in accordance with standard usage and convention.

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which is sometimes also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using a measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild-type protein and a mutein thereof. See, e.g., GCG Version 6.1.

A useful algorithm when comparing a particular polypeptide sequence to a database containing a large number of sequences from different organisms is the computer program BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62. The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences. Pearson, Methods Enzymol. 183:63-98 (1990) (incorporated by reference herein). For example, percent sequence identity between amino acid sequences can be determined using FASTA with its default parameters (a word size of 2 and the PAM250 scoring matrix), as provided in GCG Version 6.1, herein incorporated by reference.

Throughout this specification and claims, the word “comprise” or variations such as “comprises” or “comprising,” will be understood to imply the inclusion of a stated integer or group of integers but not the exclusion of any other integer or group of integers.

The term “fiber” as defined herein refers to a molded body that has the form of a filament.

The term “wet spinning” as used herein refers to a method of forming fibers from a polymer wherein the polymer is dissolved in solution and extruded into a substance that makes the dissolved polymer coagulate.

The term “coagulation bath” as used herein refers to a liquid bath comprising a substance that makes fibers coagulate.

The term “drawing” as used herein with reference to a fiber refers to the application of force to stretch a wet-spun fiber along its longitudinal axis after extrusion of the fiber into a coagulation bath. The term “undrawn fibers” refers to fibers that have been extruded into a coagulation bath but have not been subject to any drawing. The term “draw ratio” is a term of art commonly defined as the ratio between the collection rate and the feeding rate. At constant volume, it can be determine from a ratio of the initial diameter (Dᵢ) and final diameter (D_{f}) of the fiber (i.e., Dᵢ/D_{f}).

The term “glass transition temperature” as used herein refers to the temperature at which a substance transitions from a hard, rigid or “glassy” state into a more pliable, “rubbery” state.

The term “melting temperature” as used herein refers to the temperature at which a substance transitions from a rubbery state to a less-ordered liquid phase. As used herein, the term melting temperature does not refer to the temperature at which recombinant proteins containing beta sheets are denatured.

The term “plasticizer” as used herein refers to any molecule that interacts with a polypeptide sequence to prevent the polypeptide sequence from forming tertiary structures and bonds and/or to increase the mobility of the polypeptide sequence.

The term “blend” and “bi-constituent blend” as used herein refers to a composition containing multiple polymers. A “blend fiber” refers to a fiber containing recombinant spider silk polypeptide powder and cellulose.

Exemplary methods and materials are described below, although methods and materials similar or equivalent to those described herein can also be used in the practice of the present disclosure and will be apparent to those of skill in the art. All publications and other references mentioned herein are incorporated by reference in their entirety. In case of conflict, the present specification, including definitions, will control. The materials, methods, and examples are illustrative only and not intended to be limiting.

### 6.1. Recombinant Silk Proteins

Embodiments of the present disclosure include fibers synthesized from synthetic proteinaceous copolymers (i.e., recombinant polypeptides). In some embodiments, the recombinant spider silk polypeptides are based on recombinant spider silk protein fragment sequences derived from MaSp2, such as from the species Argiope bruennichi. In some embodiments, the synthesized fiber contains protein molecules that include two to twenty repeat units, in which a molecular weight of each repeat unit is greater than about 20 kDa. Within each repeat unit of the copolymer are more than about 60 amino acid residues that are organized into a number of “quasi-repeat units.” In some embodiments, the repeat unit of a polypeptide described in this disclosure has at least 95% sequence identity to a MaSp2 dragline silk protein sequence.

The repeat unit of the proteinaceous block copolymer that forms fibers with good mechanical properties can be synthesized using a portion of a silk polypeptide. These polypeptide repeat units contain alanine-rich regions and glycine-rich regions, and are 150 amino acids in length or longer. Some exemplary sequences that can be used as repeats in the proteinaceous block copolymers of this disclosure are provided in in co-owned PCT Publication WO 2015/042164, incorporated by reference in its entirety, and were demonstrated to express using a Pichia expression system.

In some embodiments, the quasi-repeat unit of the polypeptide are described by the formula {GGY-[GPG-X₁]ₙ₁-GPS-(A)ₙ₂}, where X₁ is independently selected from the group consisting of SGGQQ, GAGQQ, GQGPY, AGQQ and SQ, n1 is a number from 4 to 8, and n2 is a number from 6 to 20. The repeat unit is composed of multiple quasi-repeat units. In additional embodiments, 3 “long” quasi repeats are followed by 3 “short” quasi-repeat units. As mentioned above, short quasi-repeat units are those in which n1=4 or 5. Long quasi-repeat units are defined as those in which n1=6, 7 or 8. In some embodiments, all of the short quasi-repeats have the same X₁ motifs in the same positions within each quasi-repeat unit of a repeat unit. In some embodiments, no more than 3 quasi-repeat units out of 6 share the same X₁ motifs.

In additional embodiments, a repeat unit is composed of quasi-repeat units that do not use the same X₁ more than two occurrences in a row within a repeat unit. In additional embodiments, a repeat unit is composed of quasi-repeat units where at least 1, 2, 3, 4, 5, 6, 7, 8, 9, 10, 11, 12, 13, 14, 15, 16, 17, 18, 19 or 20 of the quasi-repeats do not use the same X₁ more than 2 times in a single quasi-repeat unit of the repeat unit.

In some embodiments, the recombinant spider silk polypeptide comprises the polypeptide sequence of SEQ ID NO: 1 (i.e., 18B). In some embodiments, the repeat unit is a polypeptide comprising SEQ ID NO: 2. These sequences are provided in Table 1:

TABLE 1
Exemplary polypeptides sequences of recombinant
protein and repeat unit

| SEQ ID | Polypeptide Sequence |
|---|---|
| SEQ ID | GGYGPGAGQQGPGSGGQQGPGGQGPYGSGQQGPGGAGQQGPGGQGPYGAAAAAAAAAG |
| NO: 1 | GYGPGAGQQGPGGAGQQGPGSQGPGGQGPYGPGAGQQGPGSQGPGSGGQQGPGGQGPYGP |
| | SAAAAAAAAAGGYGPGAGQRSQGPGGQGPYGPGAGQQGPGSQGPGSGGQQGPGGQGPYGP |
| | SAAAAAAAAGGYGPGAGQQGPGSQGPGSGGQQGPGGQGPYGPGAAAAAAAVGGYGPGAGQ |
| | QGPGSQGPGSGGQQGPGGQGPYGPSAAAAAAAAGGYGPGAGQQGPGSQGPGSGGQQGPGG |
| | QGPYGPSAAAAAAAAGGYGPGAGQQGPGSGGQQGPGGQGPYGSGQQGPGGAGQQGPGGQG |
| | PYGPGAAAAAAAAAGGYGPGAGQQGPGGAGQQGPGSQGPGGQGPYGPGAGQQGPGSQGPG |
| | SGGQQGPGGQGPYGPSAAAAAAAAAGGYGPGAGQRSQGPGGQGPYGPGAGQQGPGSQGPG |
| | SGGQQGPGGQGPYGPSAAAAAAAAGGYGPGAGQQGPGSQGPGSGGQQGPGGQGPYGPGAA |
| | AAAAAVGGYGPGAGQQGPGSQGPGSGGQQGPGGQGPYGPSAAAAAAAAGGYGPGAGQQGP |
| | GSQGPGSGGQQGPGGQGPYGPSAAAAAAAAGGYGPGAGQQGPGSGGQQGPGGQGPYGSGQ |
| | QGPGGAGQQGPGGQGPYGPGAAAAAAAAAGGYGPGAGQQGPGGAGQQGPGSQGPGGQGPY |
| | GPGAGQQGPGSQGPGSGGQQGPGGQGPYGPSAAAAAAAAAGGYGPGAGQRSQGPGGQGPY |
| | GPGAGQQGPGSQGPGSGGQQGPGGQGPYGPSAAAAAAAAGGYGPGAGQQGPGSQGPGSGG |
| | QQGPGGQGPYGPGAAAAAAAVGGYGPGAGQQGPGSQGPGSGGQQGPGGQGPYGPSAAAAA |
| | AAAGGYGPGAGQQGPGSQGPGSGGQQGPGGQGPYGPSAAAAAAAA |
| SEQ ID | GGYGPGAGQQGPGSGGQQGPGGQGPYGSGQQGPGGAGQQGPGGQGPYGPGAAAAAAAAAG |
| NO: 2 | GYGPGAGQQGPGGAGQQGPGSQGPGGQGPYGPGAGQQGPGSQGPGSGGQQGPGGQGPYGP |
| | SAAAAAAAAAGGYGPGAGQRSQGPGGQGPYGPGAGQQGPGSQGPGSGGQQGPGGQGPYGP |
| | SAAAAAAAAGGYGPGAGQQGPGSQGPGSGGQQGPGGQGPYGPGAAAAAAAVGGYGPGAGQ |
| | QGPGSQGPGSGGQQGPGGQGPYGPSAAAAAAAAGGYGPGAGQQGPGSQGPGSGGQQGPGG |
| | QGPYGPSAPA |

In some embodiments, the structure of fibers formed from the described recombinant spider silk polypeptides form beta-sheet structures, beta-turn structures, or alpha-helix structures.

In some embodiments, the secondary, tertiary and quaternary protein structures of the formed fibers are described as having nanocrystalline beta-sheet regions, amorphous beta-turn regions, amorphous alpha helix regions, randomly spatially distributed nanocrystalline regions embedded in a non-crystalline matrix, or randomly oriented nanocrystalline regions embedded in a non-crystalline matrix. While not wishing to be bound by theory, the structural properties of the proteins within the spider silk are theorized to be related to fiber mechanical properties. Crystalline regions in a fiber have been linked with the tensile strength of a fiber, while the amorphous regions have been linked to the extensibility of a fiber. The major ampullate (MA) silks tend to have higher strengths and less extensibility than the flagelliform silks, and likewise the MA silks have higher volume fraction of crystalline regions compared with flagelliform silks. Furthermore, theoretical models based on the molecular dynamics of crystalline and amorphous regions of spider silk proteins, support the assertion that the crystalline regions have been linked with the tensile strength of a fiber, while the amorphous regions have been linked to the extensibility of a fiber. Additionally, the theoretical modeling supports the importance of the secondary, tertiary and quaternary structure on the mechanical properties of RPFs. For instance, both the assembly of nano-crystal domains in a random, parallel and serial spatial distributions, and the strength of the interaction forces between entangled chains within the amorphous regions, and between the amorphous regions and the nano-crystalline regions, influenced the theoretical mechanical properties of the resulting fibers.

### 6.2. Characterization of Recombinant Spider Silk Polypeptide Powder Impurities

Different recombinant spider silk polypeptides have different physiochemical properties such as melting temperature and glass transition temperature based on the strength and stability of the secondary and tertiary structures formed by the proteins. Silk polypeptides form beta sheet structures in a monomeric form. In the presence of other monomers, the silk polypeptides form a three-dimensional crystalline lattice of beta sheet structures. The beta sheet structures are separated from, and interspersed with, amorphous regions of polypeptide sequences.

Beta sheet structures are extremely stable at high temperatures—the melting temperature of beta-sheets is approximately 257° C. as measured by fast scanning calorimetry. See Cebe et al., Beating the Heat—Fast Scanning Melts Silk Beta Sheet Crystals, Nature Scientific Reports 3:1130 (2013). As beta sheet structures are thought to stay intact above the glass transition temperature of silk polypeptides, it has been postulated that the structural transitions seen at the glass transition temperature of recombinant silk polypeptides are due to increased mobility of the amorphous regions between the beta sheets.

Plasticizers lower the glass transition temperature and the melting temperature of silk proteins by increasing the mobility of the amorphous regions and potentially disrupting beta sheet formation. Suitable plasticizers used for this purpose include water, polyalcohols (polyols) and urea. As hydrophilic portions of silk polypeptides can bind ambient water present in the air as humidity, bound ambient water may plasticize silk polypeptides.

In addition, in instances where recombinant spider silk polypeptides are produced by fermentation and recovered as recombinant spider silk polypeptide powder from the same, there may be impurities present in the recombinant spider silk polypeptide powder that act as plasticizers or otherwise inhibit the formation of tertiary structures. For example, residual lipids and sugars may act as plasticizers and thus influence the glass transition temperature of the protein by interfering with the formation of tertiary structures.

Various well-established methods may be used to assess the purity and relative composition of recombinant spider silk polypeptide powder. Size Exclusion Chromatography separates molecules based on their relative size and can be used to analyze the relative amounts of recombinant spider silk polypeptide in its aggregate and monomeric forms as well as the amount of high, low and intermediate molecular weight impurities in the recombinant spider silk polypeptide powder. Similarly, Rapid High Performance Liquid Chromatography may be used to measure various compounds present in a solution such as monomeric forms of the recombinant spider silk polypeptide. Ion Exchange Liquid Chromatography may be used to assess the concentrations of various trace molecules in solution, including impurities such as lipids and sugars. Other methods of chromatography and quantification of various molecules such as mass spectrometry are well established in the art.

In some embodiments, purity of the recombinant spider silk polypeptide powder is calculated based on the amount of the recombinant spider silk polypeptide in is monomeric and aggregate forms by weight relative to the other components of the recombinant spider silk polypeptide powder. In various instances, the purity can range from 50% by weight to 90% by weight, depending on the type of recombinant spider silk polypeptide and the techniques used to recover, separate and post-process the recombinant spider silk polypeptide powder. Depending on the embodiment, the amount by weight of recombinant spider silk polypeptide present in the recombinant spider silk polypeptide powder (i.e. the purity of the recombinant spider silk polypeptide powder) ranges from 50%-100%, from 50%-90%, from 50-80%, from 50%-70%, from 50-60%, from 60%-100%, from 60%-90%, from 60-80%, or from 60%-70%. In some specific embodiments, the purity of the recombinant spider silk polypeptide powder is approximately 50-60%.

### 6.3. Dope Rheology and Secondary Structures

Rheology is commonly used in fiber spinning to analyze the physio-chemical characteristics of material that is spun into fiber such as polymers. Different rheological characteristics may impact the ability to spin material into fiber and the mechanical characteristics of the spun fiber. Rheology can be also used to indirectly study the secondary and tertiary structures formed by recombinant spider silk polypeptides and/or cellulose under different temperatures and conditions. Depending on the embodiment, shear rheometers and/or extensional rheometers may be used to analyze different rheological properties by oscillatory and extensional rheology.

In some embodiments, small amplitude oscillatory shear (SAOS) rheology is used to measure various rheological properties including but not limited to the loss tangent (G″/G′), complex viscosity (η*) and phase angle (δ). In these embodiments, a SAOS rheometer outputs a stress response as a function of oscillation frequency, w, which can be broken down into elastic and viscous contributions. The elastic component, the solid-like behavior, is measured by the storage modulus (or elastic modulus), G′(ω), while the viscous component, the fluid-like behavior, is measured by loss modulus (or viscous modulus), G″(ω). The rheometer also measures the ratio of G″/G′, called the loss tangent, or tan (δ), which describes the extent to which the complex fluid is liquid-like (tan (δ)>>1) or solid-like (tan (δ)<<1). The rheometer outputs the values of the phase angle, δ, which spans from 90° (ideal liquid) to 0° (ideal solid). At G′ and G″ crossover, δ is 45°, and the material is transitioning from being more liquid-like to more solid-like. In addition, the complex viscosity η*, defined by η*=G*(ω)/ω, is also measured by the rheometer. In embodiments where the silk polypeptide is a recombinant silk protein that is spun into fiber, different rheological characteristics such as complex viscosity, loss tangent, and phase angle may be assessed based on a spin dope comprising the recombinant spider silk polypeptide dissolved into an appropriate solvent.

Depending on the embodiment, various rheology metrics may be used to determine whether a spin dope comprising recombinant spider silk polypeptide powder and blends thereof with cellulose is suitable for wet spinning. For example, in some embodiments, depending on the ratio of recombinant spider silk polypeptide powder and cellulose in the spin dope, a complex viscosity as measured at 10 Hz of less than 100 Pa s, less than 90 Pa s, less than 80 Ps s, less than 70 Pa s, less than 60 Pa s, less than 50 Pa s, less than 40 Pa s, less than 30 Pa s, less than 25 Pa s, less than 20 Pa s, less than 15 Pa S can indicate that a spin dope comprising recombinant spider silk polypeptide powder (and blends thereof with cellulose), is not suitable for wet spinning. Similarly, depending on the ratio of recombinant spider silk polypeptide powder and cellulose in the spin dope, in some embodiments, a complex viscosity as measured at 10 Hz of higher than 70 Pa S, higher than 80 Pa S, higher than 90 Pa S, higher than 100 Pa S can indicate that a spin dope comprising recombinant spider silk polypeptide powder (and optionally cellulose) is not suitable for wet spinning. In some embodiments, the phase angle of the spin dope comprising recombinant spider silk polypeptide may between 20-60°, 20-70°, 20-90°, 50-90°, 55-85°, 65-85°, 65-80°, 70-80°, 70-85°, 65-70°, or 50-65°.

In some embodiments, Differential Scanning calorimetry is used to determine the glass transition temperature of the recombinant spider silk polypeptide powder and/or fiber containing the same, wither as a single constituent fiber or as a bi-constituent blend with cellulose. In a specific embodiment, Modulated Differential Scanning calorimetry is used to measure the glass transition temperature.

Depending on the embodiment and the type of recombinant spider silk polypeptide powder, the glass transition temperature may have range of values. However, a measured glass transition temperature that is much lower that is typically observed for a recombinant spider silk polypeptide in its solid form may indicate that impurities or the presence of other plasticizers.

In addition, Fourier Transform Infrared (FTIR) spectroscopy data may be combined with rheology data to provide both direct characterization of tertiary structures in the recombinant spider silk polypeptide powder and/or spin dope containing the same. FTIR can be used to quantify secondary structures in recombinant spider silk polypeptide powder and/or dope comprising the silk polypeptides as discussed below in the section entitled “Fourier Transform Infrared (FTIR) Spectroscopy.”

Depending on the embodiment, FTIR may be used to quantify beta-sheet structures present in the recombinant spider silk polypeptide powder and/or spin dope containing the same. In addition, in some embodiments, FTIR may be used to quantify impurities such as sugars and lipids present in the recombinant spider silk polypeptide powder. However, various chaotropes and solubilizers used in different protein pre-processing methods may diminish the number of tertiary structures in recombinant spider silk polypeptide powder or spin dope containing the same. Accordingly, there may be no correspondence between the amount of beta sheet structures in recombinant spider silk polypeptide powder before and after is it spun into fiber. Similarly, there may be little to no correspondence between the glass transition temperature of a recombinant spider silk polypeptide powder before and after it is spun into fiber.

In some embodiments, rheological data characterizing the recombinant spider silk polypeptide powder may be combined with FTIR to analyze secondary and tertiary structures formed in by the recombinant spider silk polypeptides. In a specific embodiment, rheological data may be captured in conjunction with FTIR spectra. For exemplary methods of combining rheology and FTIR, see Boulet-Audet et al., Silk protein aggregation kinetics revealed by Rheo-IR, Acta Biomaterialia 10:776-784(2014), the entirety of which is herein incorporated by reference.

Similarly, various methods of characterizing impurities in the recombinant spider silk polypeptide powder may be combined with rheological and/or FTIR data to analyze the relationship between the presence of impurities and the formation of secondary and/or tertiary structures.

### 6.4. Spinning Recombinant Spider Silk Proteins and Cellulose Fibers

Depending on the embodiment, a blend of recombinant spider silk polypeptide powder and cellulose may be spun into fiber (“blend fiber”) using various established methods including wet spinning and dry-wet spinning. Wet spinning, as used herein, refers to extruding blend fiber produced from a spin dope into a coagulation bath or water bath. Dry-wet spinning, as used herein, refers to spinning blend fiber extruded from a spin dope into a coagulation or water bath, where the fiber passes through an air gap before entering a coagulation bath or water bath.

In most wet spinning and dry-wet spinning embodiments, a mixture of recombinant spider silk polypeptide powder and cellulose is dissolved to form a spin dope. Suitable solvents for use in a spin dope include but are not limited to: N-Methylmorpholine N-oxide (NMMO, or 4-methylmorpholine 4-oxide) monohydrate and various ionic liquids known in the art to dissolve cellulose such as 1-Ethyl-3-methylimidazolium acetate. In various embodiments, the solvent may be selected based on its ability to dissolve cellulose and effectively solubilize recombinant silk polypeptide powder. Depending on the solvent used, various salts may be added to the spin dope. In embodiments where NMMO monohydrate is used as a solvent, propyl gallate may be added as a radical scavenger.

In various embodiments, the concentration of solvent, recombinant spider silk polypeptide powder and cellulose in the spin dope can be varied based on the properties of the recombinant spider silk polypeptide, the type of cellulose used, the type of solvent used and the desired properties of the blend fiber. Concentrations may be adjusted in part based on rheological data such as the complex viscosity or the phase angle. In specific embodiments where NMMO monohydrate is used to dissolve recombinant spider silk polypeptide powder comprising the 18B protein (SEQ ID NO: 1) (hereinafter “18B powder”) and cellulose, various ratios (by weight) of 18B powder to cellulose may be used including but not limited to 100% cellulose (by weight); 10% 18B: 90% cellulose (by weight), 33% 18B: 67% cellulose (by weight); 50% 18B: 50% cellulose (by weight); 75%18B: 25% cellulose (by weight); 67% 18B: 33% cellulose (by weight); and 100% 18B (by weight). Depending on the ratio of 18B powder and cellulose, suitable concentrations of 18B powder and/or cellulose by weight in the spin dope range from: 5-60% by weight, 10-60% by weight, 10-50% by weight, 10-40% by weight, 20-40% by weight, 20-60% by weight, or 20-50% by weight.

In various embodiments, different types of cellulose may be used to create a blend fiber. Suitable types of cellulose may include: microcrystalline cellulose, alpha cellulose and dissolving wood pulp, also referred to in the art as “dissolving cellulose.” Other suitable types of cellulose will be known to those skilled in the art.

In some embodiments, a plasticizer will be added to the spin dope. Suitable plasticizers include water, polyols (e.g glycerol), lactic acid, methyl hydroperoxide, ascorbic acid, 1,4-dihydroxybenzene (1,4 Benzenediol) Benzene-1,4-diol, phosphoric acid, ethylene glycol, propylene glycol, triethanolamine, acid acetate, propane-1,3-diol or any combination thereof. In these embodiments, any of the aforementioned plasticizers would be selected based on its compatibility and safety with the spin dope solvent.

In some embodiments, various agents may be added to the spin dope to alter the rheological characteristics of the spin dope such as elongational viscosity, shear viscosity and linear viscoelasticity. Suitable agents used to alter the elongational viscosity include polyethylene glycol (PEG), Tween, Sodium dodecyl sulfate, polyethylene oxide, or any combination thereof. Other suitable agents are well known in the art.

In various embodiments, the spin dope may be subject to mixing or agitation to ensure a homogeneous spin dope. Suitable methods of mixing the spin dope include but are not limited to: centrifugal mixers, high-shear mixers, kneader-reactor mixers, impeller mixers, thin film evaporator mixers, and twin screw mixing. Other mixing methods are well established in the art.

Depending on the required initial denier of the extruded blend fiber, spin dope comprising the recombinant silk polypeptide powder may be extruded through spinnerets with varying orifice sizes. In most embodiments, the orifice will range from 50-200 μm, 50-100 μm, 50-150 μm, 100-150 μm, 100-200 μm or 150-200 μm. In some embodiments, the ideal orifice size will be based on the final draw ratio of the blend fiber, rheological properties of the dope, die swell, processing conditions (e.g. extrusion rate) and the desired linear density of the blend fiber. For example, a higher initial denier of an extruded blend fiber may be subject to a higher draw ratio than a smaller initial denier extruded blend fiber.

In some embodiments where the extruded blend fiber is dry-wet spun or wet spun, the spin dope will be heated prior to extrusion. Depending on the embodiment and the ratio of cellulose and recombinant silk protein present in the spin dope, the ideal temperature may vary. Suitable temperatures for extrusion may comprise 70-90° C., 75-85° C. and/or 80-85° C.

In embodiments where the extruded blend fiber is dry-wet spun, various sizes of air gaps may be used. In some embodiments, the air gap will depend on whether the fiber is being extruded into a water bath or a coagulation bath comprising alcohol. Suitable air gaps for extrusion into a water bath include but are not limited to at least 3 mm, at least 5 mm, at least 10 mm, at least 55 mm, at least 80 mm, or at least 100 mm. Suitable air gaps for extrusion into a coagulation bath comprising alcohol include but are not limited to at least 3 mm, at least 5 mm, at least 10 mm, at least 55 mm, at least 80 mm, or at least 100 mm.

In various embodiments, different water baths and/or coagulation baths may be used, alone or sequentially. In some embodiments, the blend fiber will be extruded into a water bath. In some embodiments, alcohol such as ethanol or methanol will be used as a coagulation agent to precipitate or otherwise coagulate the extruded spin dope. Suitable alcohols for this purpose include ethanol, methanol or any combination thereof. For example, suitable coagulation bath could contain 80% ethanol and 20% methanol; 60% ethanol and 40% methanol; 40% ethanol and 60% methanol; or 20% ethanol and 80% methanol.

In some embodiments, the coagulation bath will combine a coagulation agent with a plasticizer such as water or any of the plasticizers listed above. In a specific embodiment, the coagulation bath will comprise 50% alcohol (e.g. ethanol, methanol or the above-discussed combinations thereof) and 50% H₂0.

Depending on the embodiment, the blend fiber may be subject to any number of water and/or coagulation baths, in any order. In some embodiments, the blend fiber may be the following series of water and/or coagulation baths: a coagulation bath comprising the solvent used for dope spinning, a coagulation bath comprising only alcohol, and a coagulation bath comprising a plasticizer.

Depending on the embodiment, the total residence time in the one or more water and/or coagulation baths will range from 5-10 seconds, 5-25 seconds, 10-25 seconds, 20-50 seconds, from 25-50 seconds, from 30-50 seconds, from 35-50 seconds, from 40-50 seconds, from 20-40 seconds, from 20-35 seconds, from 20-30 seconds, and/or from 30-40 seconds. In most embodiments, the residence time will be sufficient to eliminate most or all residual solvent from the blend fiber. In some instances, the dope solvent will be recovered from the bath for re-use.

In addition to coagulation baths, the blend fiber may be subject to one or more “drips” where a small volume of liquid is applied to a blend fiber before or after it is transferred between coagulation baths. Depending on the embodiment, the liquid used for the drip may be any water, alcohol (e.g. methanol, ethanol or combinations thereof) or a combination of water and alcohol. Depending on the configuration of the drip and the spin line, the duration of time that the blend fiber is subject to the drip can range from 5-10 seconds, 5-25 seconds, 10-25 seconds, 20-50 seconds, from 25-50 seconds, from 30-50 seconds, from 35-50 seconds, from 40-50 seconds, from 20-40 seconds, from 20-35 seconds, from 20-30 seconds, and/or from 30-40 seconds

### 6.5. Drawing Fiber

Precursor blend fiber may be also drawn in order to increase the orientation of the fiber and promote three-dimensional crystalline structure. The application of force in drawing promotes molecules to align on the axis of the fiber. Polymeric molecules such as polypeptides are partially aligned when forced to flow through the spinneret orifice.

In most embodiments, the extruded blend fiber will be drawn by a first godet after it is extruded. This drawing is referred to herein as the “jet stretch.” The jet stretch of the blend fiber is calculated as a function of the extrusion rate and the take-up rate of the first godet that it is wound on. Specifically, the jet stretch is calculated as the speed of the first godet or winder that is used to take-up the extruded fiber (referred to in the examples section as “Godet 1”) divided by the Extrusion Velocity. Extrusion Velocity is calculated as extrusion rate per spinneret divided by spinneret cross session area (¼*3.1416*D²), where D is the spinneret diameter.

In some embodiments, the extruded blend fiber will not be subject to any drawing after the initial jet stretch and while it is transferred through the one or more coagulation baths and/or water baths using one or more godets to take up and transfer fiber. In other words, the extruded fiber will only be subject to the minimal amount of force necessary to move the fiber through the coagulation bath and collect fiber on the godets and the take-up rate of all of the godets used to transfer the fiber will largely be substantially the same. In other embodiments, the extruded blend fiber will be subject to additional drawing as it is moved through the one or more coagulation baths and/or water baths. Drawing can be calculated based on the draw ratio which measures the difference between the take-up rates of the various godets and winders used to transfer blend fiber between coagulation and/or water baths and wind the blend fiber on a spool for later processing.

### 6.6. Using Air Flow to Dry Fibers

In some embodiments, a multi-orifice spinneret may be used to concurrently wet spin a plurality of fibers (also referred to herein as a “tow of fibers”). Depending on the embodiment, the number of orifices in the spinneret and the corresponding number of fibers in the tow of fibers can range from 3-5, 3-10, 3-20, 5-100, 20-100, 20-80, 30-80, 20-60.

In some embodiments, the precursor fiber(s) will be subject to an air flow in order to dry the precursor fiber(s) as it exits the water bath and/or coagulation bath. Subjecting the fiber(s) to an airflow can cause plasticizers such as water and any alcohol present in the fiber(s) to evaporate from the fiber(s). In some embodiments, the air flow will be a turbulent air flow. In other embodiments, the air flow will be laminar or non-turbulent. Many different types or airflows may be combined in any order to dry the precursor fiber(s).

Various embodiments may produce a blend fiber that has a sufficient coefficient of friction to form mechanical interactions with itself or other blend fibers, making it suitable for use as a staple fiber in creating a staple-based yarn. Depending on the embodiments, the coefficient of friction of the fiber can be measured in different ways. In a specific embodiment, the coefficient of friction will be measured according to the ASTM 3808 standard. The coefficient of friction and/or roughness of the fiber may also be visually assessed using microscopy.

The coefficient of friction of the fiber impacts the ability of the fiber to form mechanical interactions (i.e. entanglement) with other fibers to form a fiberweb. The ability of the fiber to interact with other fibers to form a fiberweb may be measured in several different ways. Carding is the mechanical process used to disentangle and intermix fiber into a continuous fiberweb. Fiber that does not have the ability to form a fiberweb through carding is extruded from the carding machine as waste product. Accordingly, one method of assessing the utility of a fiber for forming a fiberweb is to assess the amount of waste that is produced by the carding process.

In other embodiments, the utility of a fiber for forming a fiberweb may be measured by determining the thickness of the fiberweb extruded from the carding machine. In other embodiments, the fiberweb may be assessed using microscopy. Suitable methods of assessing the utility of a fiber in forming a fiberweb are discussed in detail in Doguc et al., Influence of Fiber Type on Fiberweb Properties in High-Speed Carding, International Nonwovens Journal, 13(2):48-53 (2004) the entirety of which is herein incorporated by reference.

Fiber that is capable of carding and forming a fiberweb may be used to create staple or spun yarns. A staple yarn is a yarn that is comprised a number of staple fibers that have a limited length. Staple fibers may be created by cutting or chopping continuous extruded fibers. Staple yarn is created by taking the fiberweb that is output from the carding process (referred to as “sliver”) and then twisting the fiber into yarn. Depending on the embodiment, there may be a number of post-processing steps use to process the sliver such as pin drafting or combing the sliver. The staple yarn can then be used to form different garments or in other objects which incorporate textiles (e.g. upholstery).

Alternately, fibers may be used as filament yarns. Filament yarns are created by twisting one or more fibers around a common axis. Exemplary methods of making and using filament yarns and staple yarns are discussed in U.S. Patent Publication No. 2018/0216260, titled “Recombinant protein fiber yarns with improved properties,” published Aug. 2, 2018, the entirety of which is herein incorporated by reference.

In various embodiments, the blend fiber may have improved commercial characteristics relative to a fiber comprising cellulose or recombinant spider silk polypeptide powder on its own. As discussed below, the blend fiber may have a reduced shrinkage relative to fiber comprising recombinant spider silk polypeptide powder. In some instances, the blend fiber may have a superior luster, drape or hand-feel relative to fiber comprising cellulose on its own.

### 6.7. Post-Processing Fiber

Various methods of post-processing may be employed to improve the molecular alignment of the blend fiber. Depending on the amount of cellulose present in a blend fiber comprising a blend of recombinant spider silk protein and cellulose, the fiber may be heat treated (e.g. annealed using steam or heat). However, in embodiments, where a high amount of cellulose is used in the blend fiber, use of heat would be undesirable as it may damage or burn cellulose. In other instances, the fiber may be treated with various solvents to anneal the fiber and improve crystallinity of the recombinant spider silk protein (e.g. 18B protein) and cellulose in the fiber. In some instances, the blend fiber is annealed using an alcohol such as methanol. In a specific embodiment, the blend fiber is annealed using alcohol vapor.

In some embodiments, the recombinant spider silk protein will have a hydrophobic component that causes fiber comprising recombinant spider silk protein to shrink or contract upon treatment with water. Contraction of recombinant spider silk proteins is discussed at length in U.S. Patent Publication No. 2018/0282937, titled “Supercontracting fiber textiles,” published Oct. 4, 2018, the entirety of which is herein incorporated by reference. In some instances, inclusion of recombinant spider silk protein powder in a blend fiber with cellulose will limit contraction to a percentage less than 10%, less than 8%, less than 5%, less than 4%, less than 3%, less than 2% or less than 1%. In some instances, inclusion of recombinant spider silk in a blend fiber with cellulose will produce only negligible or un-measurable shrinkage.

In some instances, treating a fiber or a textile with one or more conditioners, lubricants, surfactants, emulsifiers, anti-cohesion agents or annealing agents before treating the fiber with water will alter the hand feel or drape of a textile after treatment with water. In a specific embodiment cyclopentasiloxane, emulsions of oil in water (with and without surfactants) or PDMS are used as conditioners. In a specific embodiment, annealing a fiber or a textile formed from a fiber with an alcohol improves the handfeel and drape of a water-treated blend fiber or textile.

Depending on embodiment, cellulose and recombinant spider silk polypeptide powder fibers (“blend fibers”) created according to the present disclosure may have varying mechanical properties. In some embodiments, the blend fibers has a tenacity of at least 10 cN/tex, at least 15 cN/tex, at least 20 cN/tex, at least 25 cN/tex, at least 30 cN/tex, at least 35 cN/tex, or at least 40 cN/tex. In some embodiments, the percentage elongation at break of the blend fibers is at least 5%, at least 6%, least 7%, at least 8%, at least 9% or at least 10%. In some instances the aforementioned tenacity or elongation at break is measured in accordance with ASTM D3822-14.

### EXAMPLES

### Example 1

### Purity of Recombinant 18B Polypeptide Powder

18B polypeptide sequences (SEQ ID NO: 1) comprising the FLAG tag were produced through various lots of large-scale fermentation, recovered and dried in powders (“18B powder”). Exemplary lots of 18B powder used to create the fibers discussed in this section are indicated in the table below in the column entitled “Source Ref.”

Reverse Phase High Performance Liquid Chromatography (“RP-HPLC”) was used to measure the amount by weight of 18B polypeptide monomer in the powder. The various lots of powder were dissolved using a 5M Guanidine Thiocyanate (GdSCN) reagent and injected onto an Agilent Poroshell 300SB C3 2.1×75 mm 5 μm column to separate constituents on the basis of hydrophobicity. The detection modality was UV absorbance of peptide bond at 215 nm (360 nm reference). The sample concentration of 18B-FLAG monomer was determined by using a lot of 18B-FLAG powder standard, for which the 18B-FLAG monomer concentration had been previously determined using Size Exclusion Chromatography (SEC-HPLC).

Table 2 (below) lists the purity of the exemplary lots of powder used. As shown below, the purity as expressed in % weight

TABLE 2
18B monomer by weight as determined by RP-HPLC

| Lot | 18B Monomer (Mass %) |
|---|---|
| 153 | 51.738 |

### Example 2

### Spin Dope Preparation and Rheology of the Spin Dopes

Various amounts of the above-discussed 18B powder and alpha cellulose (Sigma Aldrich) dissolved in NMMO monohydrate (Alfa Aesar) and Propyl Gallate (Sigma Aldrich) mixed with a custom mixer comprising a ChemGlass jacketed reaction vessel containing a stainless steel stirring shaft with 4 blades made of stainless steel heated from 100-110° C. to generate spin dopes. The vessel was further equipped with a chilled water condenser and a port for Nitrogen purging during mixing. Prior to dissolution, the 18B powder was stored in a vacuum at less than 60° C. for more than 48 hours to reduce the moisture content down to less than 4%.

A Malvern Kinexus Lab+Rotational Rheometer was used to measure the complex viscosity and the phase angle of the spin dopes. Parameters were set to a temperature of 80° C., a frequency of 100-0.1 Hz, and a strain of 1%. An interval of 3 points/decade was used to determine an average value for a given frequency.

Table 3 below includes the concentration by weight of the 18B powder in the spin dope, the complex viscosity and the phase angle as measured at 10 Hz.

TABLE 3
Spin Dope Formulation, Complex Viscosity
and Phase Angle with Alpha Cellulose

| | | | Dope Con- | | | |
|---|---|---|---|---|---|---|
| | | | centration | | Complex | Phase |
| | Cellu- | 18B | of Cellu- | Propyl | Viscosity | Angle |
| | lose | Powder | lose + 18B | Gallate | at 10 Hz | at 10 Hz |
| Dope | (wt %) | (wt %) | (wt %) | (wt %) | (Pa S) | (°) |
| 87 | 100 | 0 | 15 | 0.8 | 45.98 | 70.91 |
| 89 | 90 | 10 | 18 | 0.8 | 95.8 | 63.19 |
| 83 | 50 | 50 | 19.5 | 0.8 | 80.58 | 62.31 |
| 84 | 50 | 50 | 20 | 0.8 | 107.5 | 59.59 |
| 91 | 50 | 50 | 20 | 0.8 | 79.48 | 61.52 |
| 95 | 100 | 0 | 8 | 0.8 | 41.76 | 29.21 |
| 92 | 50 | 50 | 22 | 0.8 | 156.1 | 57.99 |
| 93 | 50 | 50 | 22 | 0.8 | 113.6 | 56.68 |
| 99 | 50 | 50 | 10 | 0.8 | 27.23 | 35.05 |
| 101 | 67 | 33 | 9 | 0.8 | 40.72 | 29.5 |
| 102 | 67 | 33 | 9 | 0.8 | 39.13 | 30.46 |
| 85 | 25 | 75 | 22 | 0.8 | 107.6 | 57.13 |

### Example 3

### Dry-Wet Spinning Conditions for Cellulose-Silk Blends

The lots of spin dope discussed in Example 2 were extruded from a 3-orifice spinneret, with each orifice having a diameter of 127 μm. The spinneret was heated to approximately 80-85° C. and the spin dope was extruded through an air gap ranging from 10 mm-80 mm into a water bath maintained at a temperature ranging from 18-20° C. The spin dope was extruded at a pressure ranging from 50 psi to 800 psi through at a rate ranging from 20 μl/minute/orifice to 40 μl/minute/orifice in order to form precursor fiber. Experiments 101 and 102 used a 10-orifice spinneret to extrude tows of 10 fibers. The remaining experiments (84, 85, 87, 89, 91, 92, 93, 95, 99) used a 3-orifice spinneret to extrude tows of 3 fibers. Experiment 85 used a drying oven maintained at a temperature of approximately 70° C.

Table 4 below lists the different conditions for the various fibers produced from the spin dope. As shown in Table 4, extrusion rate, pump pressure, air gap, air flow all varied of the different experiments. While the godet rates and winder rates varied over the different experiments, the godet and winder rates were approximately constant within an experiment, and therefore the fiber was not subject to any additional drawing aside from the initial jet stretch which also varied between experiments. In most experiments, the fibers were spun into a water bath. However, in various experiments, a drip was applied to the fiber between baths that included an alcohol (ethanol or methanol).

TABLE 4
Spinning Conditions

| | Ext. | | | | | | | | Drying | | | | | | |
|---|---|---|---|---|---|---|---|---|---|---|---|---|---|---|---|
| Dope- | Rate | Air | Pump | | | Godet 1 | Godet 2 | Godet 3 | Oven | Winder | | | | | Jet |
| Fiber | μl/min | Gap | Pressure | | Air Flow | m/min | m/min | m/min | m/min | m/min | Bath 1 | Drip 1 | Bath2 | Drip2 | Stretch |
| 84-1 | 120 | N/A | 101 | psi | Warm Air | 8.5 | 9.5 | 10 | N/A | 10.2 | Water | 50/50 | Water | 80:20 | 2.69 |
| | | | | | | | | | | | | EtOH | | EtOH | |
| | | | | | | | | | | | | water | | water | |
| 84-2 | 120 | N/A | 103 | psi | Warm Air | 8.5 | 9.5 | 10 | N/A | 10.2 | Water | 50/50 | Water | Water | 2.69 |
| | | | | | | | | | | | | EtOH | | | |
| | | | | | | | | | | | | water | | | |
| 84-3 | 120 | N/A | 105 | psi | Warm Air | 15 | 15.7 | 16.4 | N/A | 16.9 | Water | 50/50 | Water | None | 4.75 |
| | | | | | | | | | | | | EtOH | | | |
| | | | | | | | | | | | | water | | | |
| 85-1 | 120 | N/A | 137 | psi | No | 7 | 7.3 | N/A | N/A | 7.4 | Water | 50/50 | EtOH | Water | 2.22 |
| | | | | | | | | | | | | EtOH | | | |
| | | | | | | | | | | | | water | | | |
| 85-2 | 120 | N/A | 138 | psi | No | 7 | 7.3 | N/A | N/A | 7.4 | Water | 50/50 | EtOH | Water | 2.22 |
| | | | | | | | | | | | | EtOH | | | |
| | | | | | | | | | | | | water | | | |
| 85-3 | 120 | N/A | 138 | psi | Warm Air | 7 | 7.3 | N/A | N/A | 7.4 | Water | 50/50 | EtOH | 80/20 | 2.22 |
| | | | | | | | | | | | | EtOH | | EtOH | |
| | | | | | | | | | | | | water | | Water | |
| 85-4 | 120 | N/A | 139 | psi | Warm Air | 7 | 7.3 | N/A | N/A | 7.4 | Water | 50/50 | 50/50 | 80/20 | 2.22 |
| | | | | | | | | | | | | EtOH | EtOH | EtOH | |
| | | | | | | | | | | | | water | water | Water | |
| 85-5 | 120 | N/A | 140 | psi | Warm Air | 7 | 7.3 | N/A | N/A | 7.4 | Water | 50/50 | Water | 75/25 | 2.22 |
| | | | | | | | | | | | | EtOH | | MeOH | |
| | | | | | | | | | | | | water | | Water | |
| 85-6 | 120 | N/A | 145 | psi | No | 7 | 7.3 | N/A | 7.4 | N/A | Water | 50/50 | Water | 75/25 | 2.22 |
| | | | | | | | | | | | | EtOH | | MeOH | |
| | | | | | | | | | | | | water | | Water | |
| 85-7 | 120 | N/A | 146 | psi | No | 7 | 7.3 | N/A | 7.4 | N/A | Water | 50/50 | Water | Water | 2.22 |
| | | | | | | | | | | | | EtOH | | | |
| | | | | | | | | | | | | water | | | |
| 85-8 | 120 | N/A | 147 | psi | No | 7 | 7.3 | N/A | 7.4 | N/A | Water | 50/50 | Water | 80/20 | 2.22 |
| | | | | | | | | | | | | EtOH | | EtOH | |
| | | | | | | | | | | | | water | | Water | |
| 85-9 | 120 | N/A | N/A | | No | 7 | 7.3 | N/A | 7.4 | N/A | Water | 50/50 | Water | EtOH | 2.22 |
| | | | | | | | | | | | | EtOH | | | |
| | | | | | | | | | | | | water | | | |
| 85-10 | 120 | N/A | N/A | | No | 7 | 7.3 | N/A | 7.4 | N/A | Water | EtOH | Water | Drip2: | 2.22 |
| | | | | | | | | | | | | | | EtOH | |
| 85-11 | 120 | N/A | N/A | | Turbulent | 7 | 7.3 | N/A | 7.4 | N/A | Water | 50/50 | EtOH | N/A | 2.22 |
| | | | | | | | | | | | | EtOH | | | |
| | | | | | | | | | | | | water | | | |
| 87-1 | 120 | 10 mm | 53 | psi | No | 7 | 8 | N/A | 8 | 8.2 | Water | Water | Water | Water | 2.22 |
| 87-2 | 120 | 15 mm | 87 | psi | No | 8 | 9 | N/A | N/A | 9.3 | Water | Water | Water | Water | 2.53 |
| 87-3 | 120 | 10 mm | 89 | psi | Turbulent | 8 | 9 | N/A | N/A | 9.3 | Water | Water | Water | Water | 2.53 |
| 87-4 | 120 | 10 mm | 90 | psi | No | 8 | 9 | N/A | N/A | 9.3 | Water | Water | Water | Water | 2.53 |
| 87-5 | 120 | 10 mm | 91 | psi | No | 10 | 11.3 | N/A | N/A | 11.8 | Water | Water | Water | Water | 3.16 |
| 87-6 | 120 | 10 mm | 91 | psi | No | 20 | 22.6 | N/A | N/A | 23.0 | Water | Water | Water | Water | 6.33 |
| 87-7 | 120 | 10 mm | 92 | psi | No | 20 | 21 | N/A | N/A | 21.5 | Water | Water | Water | Water | 6.33 |
| 87-8 | 120 | 10 mm | 93 | psi | No | 8 | 9 | N/A | N/A | 9.3 | Water | Water | EtOH | N/A | 2.53 |
| 87-9 | 120 | 10 mm | 93 | psi | Turbulent | 8 | 9 | N/A | N/A | 9.3 | Water | Water | EtOH | N/A | 2.53 |
| 87-10 | 120 | 10 mm | 94 | psi | Turbulent | 8 | 9 | N/A | N/A | 9.3 | Water | Water | EtOH | N/A | 2.53 |
| 87-11 | 120 | 10 mm | 95 | psi | Yes | 20 | 21 | N/A | N/A | 22.1 | Water | Water | EtOH | N/A | 6.33 |
| 87-12 | 120 | 10 mm | 95 | psi | No | 20 | 21 | N/A | N/A | 22.1 | Water | Water | EtOH | N/A | 6.33 |
| 87-13 | 120 | 10 mm | 96 | psi | No | 35 | 36 | N/A | N/A | 36 | Water | Water | EtOH | N/A | 11.08 |
| 87-14 | 120 | 10 mm | 96 | psi | No | 75 | 75.06 | N/A | N/A | 75.4 | Water | Water | EtOH | N/A | 23.73 |
| 87-12 | 120 | 10 mm | 98 | psi | No | 75 | 75.06 | N/A | N/A | 75.4 | Water | Water | EtOH | N/A | 23.73 |
| 89-2 | 120 | 80 mm | 93 | psi | Warm Air | 10 | 11.5 | N/A | N/A | 12 | Water | Water | Water | Water | 3.16 |
| 89-3 | 120 | 80 mm | 93 | psi | No | 16 | 16.4 | N/A | N/A | 17 | Water | Water | Water | Water | 5.06 |
| 89-4 | 120 | 80 mm | 94 | psi | Turbulent | 16 | 16.4 | N/A | N/A | 17 | Water | Water | Water | Water | 5.06 |
| 89-5 | 120 | 10 mm | 97 | psi | Turbulent | 10 | 11.5 | N/A | N/A | 11.3 | Water | Water | Water | Water | 3.16 |
| 89-6 | 120 | 10 mm | 99 | psi | No | 10 | 11.5 | N/A | N/A | 11.3 | Water | Water | Water | Water | 3.16 |
| 89-7 | 120 | 10 mm | 104 | psi | No | 10 | 10.8 | N/A | N/A | 11.3 | Water | Water | EtOH | N/A | 3.16 |
| 89-8 | 120 | 10 mm | 104 | psi | Turbulent | 10 | 10.9 | N/A | N/A | 11.3 | Water | Water | EtOH | N/A | 3.16 |
| 89-9 | 120 | 10 mm | 105 | psi | No | 35 | 36 | N/A | N/A | 37 | Water | Water | EtOH | N/A | 11.08 |
| 89-10 | 120 | 55 mm | 108 | psi | No | 35 | 36 | N/A | N/A | 37 | Water | Water | EtOH | N/A | 11.08 |
| 89-11 | 120 | 55 mm | 110 | psi | Turbulent | 34.7 | 35.3 | N/A | N/A | 36.4 | Water | Water | EtOH | N/A | 10.98 |
| 91-1 | 120 | N/A | 149 | psi | No | 8 | 9 | N/A | N/A | 9.2 | Water | 50/50 | EtOH | N/A | 2.53 |
| | | | | | | | | | | | | EtOH | | | |
| | | | | | | | | | | | | water | | | |
| 91-2 | 120 | N/A | 151 | pi | Warm air | 8 | 9 | N/A | N/A | 9.2 | Water | 50/50 | EtOH | N/A | 2.53 |
| | | | | | | | | | | | | EtOH | | | |
| | | | | | | | | | | | | water | | | |
| 91-3 | 120 | N/A | 158 | psi | Warm air | 18 | 18.3 | N/A | N/A | 18.4 | Water | 50/50 | EtOH | N/A | 5.70 |
| | | | | | | | | | | | | EtOH | | | |
| | | | | | | | | | | | | water | | | |
| 91-4 | 120 | N/A | 223 | psi | Turbulent | 8 | 8.56 | N/A | N/A | 8.9 | Water | 50/50 | EtOH | N/A | 2.53 |
| | | | | | | | | | | | | EtOH | | | |
| | | | | | | | | | | | | water | | | |
| 91-5 | 120 | N/A | 212 | psi | N/A | 30 | 30.6 | N/A | N/A | 30.9 | 50/50 | EtOH | EtOH | N/A | 9.49 |
| | | | | | | | | | | | EtOH | | | | |
| | | | | | | | | | | | water | | | | |
| 92-1 | 120 | 62 mm | 259 | psi | N/A | 8 | 9 | N/A | N/A | 9.2 | Water | 50/50 | EtOH | N/A | 2.53 |
| | | | | | | | | | | | | EtOH | | | |
| | | | | | | | | | | | | water | | | |
| 92-2 | 120 | 90 mm | 259 | | | 29 | 29.3 | N/A | N/A | 29.8 | Water | 50/50 | EtOH | N/A | 9.18 |
| | | | | | | | | | | | | EtOH | | | |
| | | | | | | | | | | | | water | | | |
| 92-3 | 120 | 90 mm | 298 | | Turbulent | 28 | 28.2 | N/A | N/A | 28.8 | Water | 50/50 | EtOH | N/A | 8.86 |
| | | | | | | | | | | | | EtOH | | | |
| | | | | | | | | | | | | water | | | |
| 92-4 | 120 | 90 mm | 330 | | Turbulent | 18 | 18.2 | N/A | N/A | 18.6 | Water | 50/50 | EtOH | N/A | 5.70 |
| | | | | | | | | | | | | EtOH | | | |
| | | | | | | | | | | | | water | | | |
| 92-5 | 120 | 90 mm | 326 | | Turbulent | 20 | 20.2 | N/A | N/A | 20.6 | Water | 50/50 | EtOH | N/A | 6.33 |
| | | | | | | | | | | | | EtOH | | | |
| | | | | | | | | | | | | water | | | |
| 92-6 | 120 | 90 mm | 325 | | Turbulent | 23 | 23.3 | N/A | N/A | 24 | Water | 50/50 | EtOH | N/A | 7.28 |
| | | | | | | | | | | | | EtOH | | | |
| | | | | | | | | | | | | water | | | |
| 92-7 | 120 | 90 mm | 319 | | Turbulent | 25 | 25.2 | N/A | N/A | 25.5 | Water | 50/50 | EtOH | N/A | 7.91 |
| | | | | | | | | | | | | EtOH | | | |
| | | | | | | | | | | | | water | | | |
| 92-8 | 120 | 90 mm | 311 | | Warm air | 24 | 24.2 | N/A | N/A | 25 | Water | 50/50 | EtOH | EtOH | 7.59 |
| | | | | | | | | | | | | EtOH | | | |
| | | | | | | | | | | | | water | | | |
| 92-9 | 120 | 90 mm | 310 | | Warm air | 24 | 24.2 | N/A | N/A | 25 | Water | 50/50 | EtOH | 50/50 | 7.59 |
| | | | | | | | | | | | | EtOH | | EtOH | |
| | | | | | | | | | | | | water | | water | |
| 92-10 | 120 | 90 mm | 310 | | Warm air | 24 | 24.2 | N/A | N/A | 25 | Water | 50/50 | EtOH | 50/50 | 7.59 |
| | | | | | | | | | | | | EtOH | | EtOH | |
| | | | | | | | | | | | | water | | water | |
| 92-11 | 120 | 90 mm | 301 | | Warm air | 24 | 24.2 | N/A | N/A | 25 | Water | 50/50 | EtOH | 50/50 | 7.59 |
| | | | | | | | | | | | | EtOH | | EtOH | |
| | | | | | | | | | | | | water | | water | |
| 92-12 | 120 | 90 mm | 300 | | Warm air | 24 | 24.2 | N/A | N/A | 25 | Water | 50/50 | EtOH | 50/50 | 7.59 |
| | | | | | | | | | | | | EtOH | | EtOH | |
| | | | | | | | | | | | | water | | water | |
| 92-13 | 120 | 90 mm | 300 | | N/A | 24 | 24.2 | N/A | N/A | 25 | Water | 50/50 | N/A | N/A | 7.59 |
| | | | | | | | | | | | | EtOH | | | |
| | | | | | | | | | | | | water | | | |
| 92-14 | 120 | 90 mm | 300 | | N/A | 24 | 24.2 | N/A | N/A | 25 | Water | 50/50 | Water | EtOH | 7.59 |
| | | | | | | | | | | | | EtOH | | | |
| | | | | | | | | | | | | water | | | |
| 93-1 | 60 | 25 mm | 93 | psi | Turbulent | 8 | 8.1 | N/A | N/A | 8.2 | Water | 50/50 | EtOH | N/A | 5.06 |
| | | | | | air flow | | | | | | | EtOH | | | |
| | | | | | | | | | | | | water | | | |
| 93-2 | 60 | 25 mm | 86 | psi | Turbulent | 18 | 18.3 | N/A | N/A | 18.9 | Water | 50/50 | EtOH | N/A | 11.39 |
| | | | | | air flow | | | | | | | EtOH | | | |
| | | | | | | | | | | | | water | | | |
| 95-1 | 120 | 40 mm | 205 | psi | N/A | 10 | 10.1 | N/A | N/A | 10.5 | Water | Water | Water | N/A | 3.16 |
| 95-2 | 120 | 40 mm | 205 | psi | N/A | 40 | 40.4 | N/A | N/A | 40.7 | Water | Water | Water | N/A | 12.66 |
| 95-3 | 60 | 40 mm | 175 | psi | N/A | 10 | 10.1 | N/A | N/A | 10.3 | Water | Water | Water | N/A | 6.33 |
| 95-4 | 80 | 40 mm | 226 | psi | N/A | 40 | 40.3 | N/A | N/A | 40.9 | Water | Water | Water | N/A | 18.99 |
| 95-5 | 80 | 40 mm | N/A | | N/A | 40 | 40.3 | N/A | N/A | | Water | Water | Water | N/A | 18.99 |
| 95-6 | 80 | 100 mm | N/A | | N/A | 20 | 20.2 | N/A | N/A | 21 | Water | Water | Water | N/A | 9.49 |
| 99-1 | 80 | 30 mm | 163 | psi | Turbulent | 12 | 12.1 | N/A | N/A | 12.5 | Water | 50/50 | Water | EtOH | 5.70 |
| | | | | | air flow | | | | | | | EtOH | | | |
| | | | | | | | | | | | | water | | | |
| 99-2 | 80 | 30 mm | 221 | psi | Warm air | 20 | 20.1 | N/A | N/A | 20.4 | Water | 50/50 | EtOH | N/A | 9.49 |
| | | | | | | | | | | | | EtOH | | | |
| | | | | | | | | | | | | water | | | |
| 99-3 | 80 | 30 mm | 254 | psi | Turbulent | 20 | 20.1 | N/A | N/A | 20.4 | Water | 50/50 | EtOH | N/A | 9.49 |
| | | | | | air flow | | | | | | | EtOH | | | |
| | | | | | | | | | | | | water | | | |
| 99-4 | 80 | 30 mm | 221 | psi | Warm air | 20 | 20.1 | N/A | N/A | 20.4 | Water | 50/50 | EtOH | N/A | 9.49 |
| | | | | | | | | | | | | EtOH | | | |
| | | | | | | | | | | | | water | | | |
| 99-5 | 80 | 30 mm | 372 | psi | Warm air | 8 | 8 | N/A | N/A | 8.4 | Water | 50/50 | 10 mm | N/A | 3.80 |
| | | | | | | | | | | | | EtOH | SDS in | | |
| | | | | | | | | | | | | water | water | | |
| 99-6 | 80 | 30 mm | 370 | psi | N/A | 18 | 18 | N/A | N/A | 18.3 | Water | 50/50 | 10 mm | N/A | 8.54 |
| | | | | | | | | | | | | EtOH | SDS in | | |
| | | | | | | | | | | | | water | water | | |
| 99-7 | 80 | 30 mm | 388 | psi | Turbulent | 18 | 18 | N/A | N/A | 18.3 | Water | 50/50 | 10 mm | N/A | 8.54 |
| | | | | | air flow | | | | | | | EtOH | SDS in | | |
| | | | | | | | | | | | | water | water | | |
| 101-1 | 60 | 70 mm | 208 | psi | N/A | 19 | 19.9 | N/A | N/A | 19.4 | Water | Water | Water | N/A | 12.03 |
| 101-2 | 90 | 70 mm | 385 | psi | N/A | 28 | 28 | N/A | N/A | 29 | Water | Water | Water | N/A | 11.81 |
| 101-3 | 90 | 30 mm | 443 | psi | N/A | 31 | 31.1 | N/A | N/A | 31.4 | Water | Water | Water | N/A | 13.08 |
| 101-4 | 90 | 30 mm | 536 | psi | N/A | 32 | 32.2 | N/A | N/A | 32.6 | Water | Water | Water | N/A | 13.50 |
| 101-5 | 90 | 30 mm | 576 | psi | N/A | 28 | 28 | N/A | N/A | 28.7 | Water | Water | Water | N/A | 11.81 |
| 101-6 | 90 | 30 mm | 576 | psi | N/A | 26 | 26 | N/A | N/A | 26.1 | Water | Water | Water | N/A | 10.97 |
| 101-7 | 90 | 30 mm | 660 | psi | N/A | 26 | 26 | N/A | N/A | 26.4 | Water | Water | Water | N/A | 10.97 |
| 101-8 | 300 | 30 mm | 368 | psi | N/A | 8 | 8 | N/A | N/A | 8.2 | Water | Water | Water | N/A | 3.38 |
| 101-9 | 300 | 30 mm | 368 | psi | N/A | 8 | 8.1 | N/A | N/A | 8.2 | Water | Water | Water | N/A | 3.38 |
| 102-1 | 200 | N/A | 253 | psi | Turbulent | 8 | 8 | N/A | N/A | 8.1 | Water | Water | Ethanol | N/A | 5.06 |
| 102-2 | 200 | N/A | 313 | psi | Turbulent | 8 | 8 | N/A | N/A | 8.1 | Water | Water | Ethanol | N/A | 5.06 |
| 102-3 | 300 | N/A | 313 | psi | Turbulent | 12 | 12 | N/A | 12.1 | 12.1 | Water | Water | Ethanol | N/A | 5.06 |
| 102-4 | 300 | N/A | 313 | psi | Turbulent | 12 | 12 | N/A | 12.1 | 12.1 | Water | Water | Ethanol | N/A | 5.06 |
| 102-5 | 200 | N/A | 313 | psi | Turbulent | 8 | 8 | N/A | N/A | 8.1 | Water | Water | Ethanol | N/A | 5.06 |
| 102-6 | 200 | N/A | 313 | psi | Turbulent | 8 | 8 | N/A | N/A | 8.1 | Water | Water | Ethanol | N/A | 5.06 |
| 102-7 | 200 | N/A | 313 | psi | Turbulent | 8 | 8 | N/A | N/A | 8.1 | Water | Water | Ethanol | N/A | 5.06 |

### Example 4

### Mechanical Properties of Spun Fibers

Mechanical properties of fiber from the above-discussed experiments were measured using a FAVIMAT model tensile test equipment model Favimat+ and Robot2 using either a single filament or a tow of at least 2 filaments. Linear density was measured in accordance with ASTM D1577. Tensile properties were measured in accordance with ASTM D3822-14. Each of the properties were measured a number of times as listed tabulated below (“# of samples”) and the mean was calculated. The following tensile properties were measured and tabulated below: tenacity, percentage elongation at break, maximum force at break, work at break and initial modulus.

Table 5 below lists the properties collected from various fiber samples formed under the conditions listed in Example 3 with the spin dopes discussed in Example 2.

TABLE 5
Mechanical properties of Cellulose/18B Fibers

| | % by | | Mean | Mean | Mean | | Mean | | Mean | |
|---|---|---|---|---|---|---|---|---|---|---|
| | weight | Single | Linear | Elongation | Elongation | Mean | Work | Mean | Initial. | |
| Dope - | Cellulose/ | Filament | Density | (Fmax) | At Break | Maximum | (break) | Tenacity | Modulus | # of |
| Fiber | 18B | or Tow | (dtex) | (%) | (%) | Force cn | cn*cm | cN/tex | cN/tex | samples |
| 95-1 | 100/0 | Single | 2.93 | 6.27 | 6.49 | 9.93 | 0.93 | 33.96 | 1759.98 | 15 |
| 95-2 | 100/0 | Tow (3) | 2.24 | 6.26 | 6.5 | 10.06 | 0.86 | 44.87 | 2095.99 | 15 |
| 95-3 | 100/0 | Tow (3) | 4.46 | 6.61 | 6.82 | 17.2 | 1.64 | 38.65 | 1940.74 | 15 |
| 95-4 | 100/0 | Tow (3) | 1.5 | 5.87 | 6.11 | 7.16 | 0.58 | 47.98 | 2226.9 | 14 |
| 95-5 | 100/0 | Tow (3) | 1.07 | 5.17 | 5.49 | 5.44 | 0.35 | 50.91 | 2192.62 | 6 |
| 95-6 | 100/0 | Tow (3) | 3.04 | 6.42 | 6.65 | 13.56 | 1.19 | 44.63 | 2028.13 | 15 |
| 087-13 | 100/0 | Single | 2.55 | 6.68 | 6.93 | 4.69 | 0.5 | 18.63 | 1149.15 | 11 |
| 087-12 | 100/0 | Single | 3.68 | 10.77 | 11.01 | 6.24 | 1.09 | 17 | 944.79 | 15 |
| 087-11 | 100/0 | Single | 3.48 | 8.14 | 8.39 | 6.45 | 0.87 | 18.57 | 1088.36 | 15 |
| 087-8 | 100/0 | Tow (3) | 23.57 | 20.52 | 23.62 | 37.38 | 12.6 | 15.87 | 758.82 | 14 |
| 087-5 | 100/0 | Tow (3) | 19.51 | 10.31 | 11.11 | 33.63 | 5.66 | 17.25 | 970.74 | 15 |
| 087-7 | 100/0 | Tow (3) | 11.09 | 6.91 | 8.14 | 19.9 | 2.18 | 17.95 | 1104.93 | 14 |
| 089-11 | 90/10 | Tow (3) | 8.12 | 6.66 | | 15.85 | 1.61 | 19.52 | 1116.57 | 11 |
| 089-10 | 90/10 | Tow (3) | 6.79 | 4.74 | 5.9 | 15.47 | 1.03 | 23.09 | 1375.77 | 15 |
| 089-9 | 90/10 | Single | 2.59 | 4.66 | 4.9 | 5.1 | 0.35 | 19.72 | 1228.12 | 15 |
| 089-8 | 90/10 | Single | 8.04 | 12.21 | 12.44 | 12.21 | 2.48 | 15.23 | 816.87 | 14 |
| 089-7 | 90/10 | Single | 7.72 | 7.51 | 7.73 | 12.85 | 1.53 | 16.67 | 991.1 | 15 |
| 089-5 | 90/10 | Tow (3) | 16.09 | 9.02 | 9.51 | 27.34 | 3.98 | 16.99 | 989.34 | 14 |
| 089-4 | 90/10 | Tow (3) | 15.18 | 8.3 | 9.24 | 27.03 | 3.59 | 17.82 | 1043.8 | 15 |
| 089-3 | 90/10 | Tow (3) | 15 | 8.14 | 8.37 | 26.62 | 3.43 | 17.79 | 1064.99 | 15 |
| 101-1 | 67/33 | Tow (10) | 2.69 | 4.75 | 4.97 | 10.45 | 0.71 | 38.81 | 2128.72 | 15 |
| 101-3 | 67/33 | Tow (10) | 2.57 | 4.88 | 5.11 | 10.39 | 0.7 | 40.48 | 2200.65 | 15 |
| 101-3 | 67/33 | Tow (10) | 1.94 | 4.25 | 4.46 | 7.95 | 0.47 | 41.15 | 2155.01 | 15 |
| 101-4 | 67/33 | Single | 0.82 | 5.79 | 6.01 | 3.25 | 0.26 | 39.72 | 2034 | 15 |
| 102-1 | 67/33 | Single | 2.56 | 4.78 | 5.01 | 7.17 | 0.5 | 28.24 | 1760.85 | 14 |
| 102-2 | 67/33 | Single | 2.23 | 7.61 | 7.83 | 6.53 | 0.75 | 29.45 | 1620.63 | 15 |
| 102-3 | 67/33 | Single | 2.23 | 4.79 | 5.01 | 6.79 | 0.46 | 30.41 | 1831.26 | 13 |
| 102-4 | 67/33 | Single | 2.33 | 4.14 | 4.37 | 6.11 | 0.36 | 26.85 | 1687.08 | 15 |
| 102-5 | 67/33 | Single | 2.09 | 4.35 | 4.59 | 5.61 | 0.36 | 26.83 | 1704.93 | 15 |
| 084-2 | 50/50 | Tow (3) | 24.77 | 5.63 | 5.87 | 38.46 | 3.43 | 15.53 | 931.77 | ? |
| 091-1 | 50/50 | Tow (3) | 9.25 | 3.79 | 4.79 | 13.33 | 0.76 | 14.5 | 947.28 | 15 |
| 091-3 | 50/50 | Tow (3) | 5.13 | 7.93 | 8.16 | 7.45 | 1 | 14.64 | 942.42 | 15 |
| 091-4 | 50/50 | Tow (3) | 15.53 | 13.76 | 14.02 | 16.7 | 3.92 | 10.76 | 608.28 | 15 |
| 091-2 | 50/50 | Single | 11.25 | 8.76 | 9.05 | 14.09 | 2.16 | 12.68 | 801.2 | 15 |
| 091-5 | 50/50 | Tow (3) | 6.52 | 3.97 | 4.21 | 8.67 | 0.52 | 13.36 | 862.64 | 15 |
| 091-2 | 50/50 | Single | 10.82 | 4.65 | 7.15 | 13.85 | 1.04 | 12.85 | 837.7 | 15 |
| 092-1 | 50/50 | Single | 11.01 | 3.68 | 4.75 | 14.23 | 0.79 | 13.14 | 827.33 | 15 |
| 092-2 | 50/50 | Single | 3.86 | 5.21 | 5.45 | 5.85 | 0.49 | 15.23 | 1003.58 | 15 |
| 092-3 | 50/50 | Single | 3.6 | 5.02 | 5.24 | 5.65 | 0.46 | 15.78 | 1023.66 | 15 |
| 092-4 | 50/50 | Single | 6.27 | 8.58 | 8.83 | 8.26 | 1.21 | 13.43 | 831.75 | 15 |
| 092-5 | 50/50 | Single | 5.03 | 4.08 | 4.62 | 7.14 | 0.48 | 14.59 | 968.94 | 15 |
| 092-6 | 50/50 | Single | 4.54 | 5.65 | 5.87 | 6.79 | 0.63 | 15.05 | 969.03 | 15 |
| 092-7 | 50/50 | Single | 4.13 | 6.03 | 6.26 | 6.07 | 0.6 | 14.88 | 961.73 | 15 |
| 092-8 | 50/50 | Single | 4.54 | 5.93 | 6.17 | 6.9 | 0.68 | 15.44 | 994.92 | 14 |
| 092-9 | 50/50 | Tow (3) | 11.35 | 3.73 | 3.97 | 19.47 | 1.07 | 17.17 | 1128.68 | 13 |
| 092-10 | 50/50 | Tow (3) | 29.06 | 7.44 | 7.68 | 35.67 | 4.95 | 12.37 | 782.37 | 13 |
| 092-11 | 50/50 | Single | 4.21 | 5.65 | 5.89 | 6.46 | 0.6 | 15.52 | 1012.3 | 14 |
| 092-12 | 50/50 | Single | 4.53 | 5.27 | 5.49 | 6.98 | 0.6 | 15.5 | 1000.08 | 15 |
| 099-3 | 50/50 | Single | 1.18 | 4.4 | 4.62 | 3.74 | 0.23 | 31.96 | 1815.43 | 15 |
| 099-1 | 50/50 | Single | 2.05 | 3.22 | 3.47 | 6.14 | 0.27 | 29.96 | 1755.13 | 12 |
| 099-2 | 50/50 | Single | 1.11 | 4.22 | 4.43 | 3.5 | 0.2 | 31.87 | 1818.05 | 15 |
| 099-5 | 50/50 | Tow (3) | 7.89 | 4.36 | 4.59 | 23.06 | 1.43 | 29.17 | 1716.95 | 15 |
| 099-6 | 50/50 | Tow (3) | 3.68 | 3.72 | 3.95 | 10.93 | 0.56 | 29.67 | 1793.58 | 12 |
| 099-7 | 50/50 | Tow (3) | 4.12 | 4.73 | 4.96 | 12.93 | 0.87 | 31.45 | 1776.48 | 15 |
| 101-1 | 67/33 | Tow (10) | 2.69 | 4.75 | 4.97 | 10.45 | 0.71 | 38.81 | 2128.72 | 15 |
| 101-3 | 67/33 | Tow (10) | 2.57 | 4.88 | 5.11 | 10.39 | 0.7 | 40.48 | 2200.65 | 15 |
| 101-3 | 67/33 | Tow (10) | 1.94 | 4.25 | 4.46 | 7.95 | 0.47 | 41.15 | 2155.01 | 15 |
| 101-4 | 67/33 | Single | 0.82 | 5.79 | 6.01 | 3.25 | 0.26 | 39.72 | 2034 | 15 |
| 085-1 | 25/75 | Tow (3) | 36.36 | 13.63 | 16.44 | 28.93 | 7.3 | 7.96 | 501.99 | 10 |
| 085-2 | 25/75 | Tow (3) | 35.69 | 2.84 | 5.01 | 34.1 | 1.28 | 9.57 | 587.32 | 10 |
| 085-7 | 25/75 | Tow (3) | 32.26 | 2.9 | 4.34 | 36.15 | 1.41 | 11.22 | 690.74 | 10 |
| 085-10 | 25/75 | Tow (3) | 36.54 | 2.73 | 5.41 | 33.53 | 1.19 | 9.17 | 559.43 | 8 |
| 085-9 | 25/75 | Tow (3) | 33.1 | 2.7 | 7.35 | 32.5 | 1.14 | 9.83 | 605.41 | 10 |

### Example 5

### Contraction of Spun Fibers

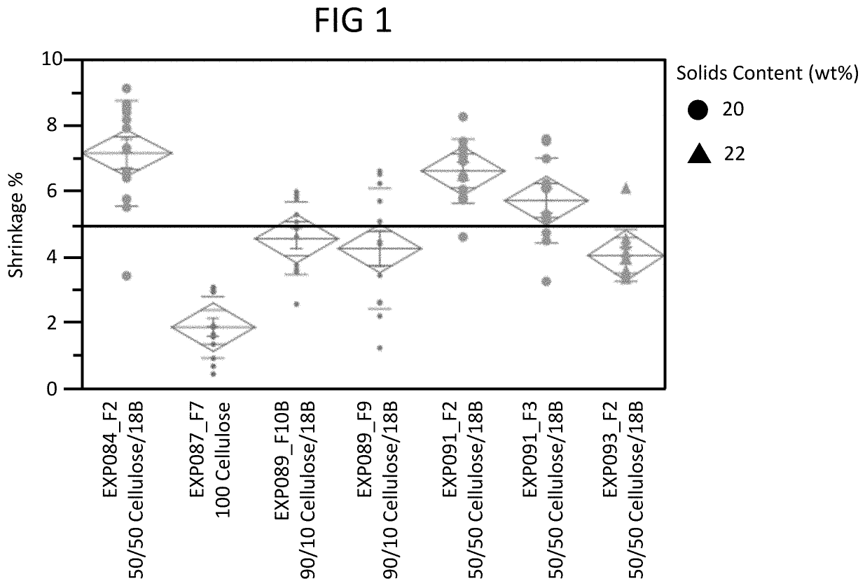

As recombinant spider silk protein is an inherently hydrophobic protein, fibers formed from recombinant spider silk protein may shrink or contract upon treatment with water. In order to investigate the effect of cellulose on shrinkage of recombinant spider silk fiber, multiple samples from the fibers described in the above discussed experiments were measured in length before and after treatment with water. Table 6 below lists the percentage shrinkage for the fibers discussed in Example 3. FIG. 1 include a graph of selected results from Table 6.

TABLE 6
Shrinkage of Blend Fibers

| Dope- | Cellulose/ | | Initial | | |
|---|---|---|---|---|---|
| Fiber | 18B | Sample | Length | Final Length | Shrinkage % |
| 87-7 | 100/0 | 1 | 55.14 | 54.28 | 1.559666304 |
| 87-7 | 100/0 | 2 | 54.56 | 52.95 | 2.950879765 |
| 87-7 | 100/0 | 3 | 52.92 | 51.37 | 2.928949358 |
| 87-7 | 100/0 | 4 | 53.1 | 52.87 | 0.4331450094 |
| 87-7 | 100/0 | 5 | 52.73 | 52.25 | 0.9102977432 |
| 87-7 | 100/0 | 6 | 53.34 | 51.72 | 3.03712036 |
| 87-7 | 100/0 | 7 | 56.11 | 55.07 | 1.85350205 |
| 87-7 | 100/0 | 8 | 55.98 | 54.26 | 3.072525902 |
| 87-7 | 100/0 | 9 | 58.21 | 57.43 | 1.339975949 |
| 87-7 | 100/0 | 10 | 56.69 | 56.31 | 0.6703122244 |
| 87-7 | 100/0 | 11 | 56.24 | 55.17 | 1.902560455 |
| 87-7 | 100/0 | 12 | 56.01 | 55.09 | 1.642563828 |
| 95-4 | 100/0 | 1 | 53.77 | 52.37 | 2.603682351 |
| 95-4 | 100/0 | 2 | 54.38 | 53.04 | 2.464141228 |
| 95-4 | 100/0 | 3 | 54.67 | 53.27 | 2.560819462 |
| 95-4 | 100/0 | 4 | 56.28 | 55.07 | 2.149964463 |
| 95-4 | 100/0 | 5 | 56.33 | 55.52 | 1.437954909 |
| 95-4 | 100/0 | 6 | 56.46 | 55.83 | 1.115834219 |
| 95-4 | 100/0 | 7 | 54.63 | 53.87 | 1.391177009 |
| 95-4 | 100/0 | 8 | 54.31 | 52.43 | 3.46160928 |
| 95-4 | 100/0 | 9 | 54.76 | 52.82 | 3.542731921 |
| 95-4 | 100/0 | 10 | 53.72 | 52.7 | 1.898734177 |
| 95-4 | 100/0 | 11 | 54.01 | 53.19 | 1.518237363 |
| 95-4 | 100/0 | 12 | 54.13 | 53.48 | 1.200812858 |
| 89-9 | 90/10 | 1 | 53.78 | 52.37 | 2.621792488 |
| 89-9 | 90/10 | 2 | 53.01 | 52.36 | 1.226183739 |
| 89-9 | 90/10 | 3 | 52.51 | 50.17 | 4.456294039 |
| 89-9 | 90/10 | 4 | 54.68 | 53.26 | 2.596927579 |
| 89-9 | 90/10 | 5 | 53.88 | 51.52 | 4.380103935 |
| 89-9 | 90/10 | 6 | 55.51 | 52.35 | 5.692667988 |
| 89-9 | 90/10 | 7 | 55.22 | 51.78 | 6.229626947 |
| 89-9 | 90/10 | 8 | 55.13 | 52.33 | 5.078904408 |
| 89-9 | 90/10 | 9 | 54.78 | 52.9 | 3.431909456 |
| 89-9 | 90/10 | 10 | 54.62 | 53.42 | 2.196997437 |
| 89-9 | 90/10 | 11 | 53.91 | 50.35 | 6.60359859 |
| 89-9 | 90/10 | 12 | 53.44 | 49.96 | 6.511976048 |
| 89-10 | 90/10 | 1 | 54.4 | 52.48 | 3.529411765 |
| 89-10 | 90/10 | 2 | 54.76 | 51.99 | 5.058436815 |
| 89-10 | 90/10 | 3 | 54.11 | 50.93 | 5.876917391 |
| 89-10 | 90/10 | 4 | 53.44 | 50.62 | 5.276946108 |
| 89-10 | 90/10 | 5 | 53.28 | 50.82 | 4.617117117 |
| 89-10 | 90/10 | 6 | 54.86 | 52.81 | 3.736784542 |
| 89-10 | 90/10 | 7 | 55.31 | 52.62 | 4.863496655 |
| 89-10 | 90/10 | 8 | 55.88 | 52.54 | 5.977093772 |
| 89-10 | 90/10 | 9 | 55.66 | 52.45 | 5.767157743 |
| 89-10 | 90/10 | 10 | 54.34 | 52.95 | 2.557968347 |
| 89-10 | 90/10 | 11 | 54 | 51.98 | 3.740740741 |
| 89-10 | 90/10 | 12 | 54.43 | 52.47 | 3.600955356 |
| 101-3 | 67/33 | 1 | 56.08 | 54.45 | 2.906562054 |
| 101-3 | 67/33 | 2 | 53.89 | 52.4 | 2.764891446 |
| 101-3 | 67/33 | 3 | 54.64 | 53.96 | 1.244509517 |
| 101-3 | 67/33 | 4 | 55.77 | 55.09 | 1.219293527 |
| 101-3 | 67/33 | 5 | 55.19 | 54.31 | 1.594491756 |
| 101-3 | 67/33 | 6 | 54.86 | 53.98 | 1.604083121 |
| 101-3 | 67/33 | 7 | 54.24 | 53.41 | 1.530235988 |
| 101-3 | 67/33 | 8 | 54.68 | 53.95 | 1.335040234 |
| 101-3 | 67/33 | 9 | 54.9 | 53.64 | 2.295081967 |
| 101-3 | 67/33 | 10 | 55.52 | 54.18 | 2.413544669 |
| 101-3 | 67/33 | 11 | 55.19 | 53.9 | 2.33737996 |
| 101-3 | 67/33 | 12 | 55.66 | 54.12 | 2.766798419 |
| 102-3 | 67/33 | 1 | 56.16 | 55.35 | 1.442307692 |
| 102-3 | 67/33 | 2 | 56.97 | 55.93 | 1.825522205 |
| 102-3 | 67/33 | 3 | 56 | 55.55 | 0.8035714286 |
| 102-3 | 67/33 | 4 | 56.87 | 56.22 | 1.142957623 |
| 102-3 | 67/33 | 5 | 56.6 | 55.51 | 1.925795053 |
| 102-3 | 67/33 | 6 | 56.9 | 55.29 | 2.829525483 |
| 102-3 | 67/33 | 7 | 55.29 | 54.23 | 1.917164044 |
| 102-3 | 67/33 | 8 | 54.87 | 53.61 | 2.296336796 |
| 102-3 | 67/33 | 9 | 54.16 | 53.27 | 1.643279173 |
| 102-3 | 67/33 | 10 | 54.98 | 53.84 | 2.073481266 |
| 102-3 | 67/33 | 11 | 54.28 | 53.12 | 2.13706706 |
| 102-3 | 67/33 | 12 | 55.74 | 54.35 | 2.493720847 |
| 84-2 | 50/50 | 1 | 52.29 | 49.28 | 5.756358768 |
| 84-2 | 50/50 | 2 | 52.95 | 48.12 | 9.121813031 |
| 84-2 | 50/50 | 3 | 54.33 | 49.77 | 8.393152954 |
| 84-2 | 50/50 | 4 | 54.24 | 50.28 | 7.300884956 |
| 84-2 | 50/50 | 5 | 53.93 | 49.99 | 7.305766735 |
| 84-2 | 50/50 | 6 | 55.19 | 50.48 | 8.534154738 |
| 84-2 | 50/50 | 7 | 50.57 | 48.84 | 3.421000593 |
| 84-2 | 50/50 | 8 | 50.16 | 46.95 | 6.399521531 |
| 84-2 | 50/50 | 9 | 51.91 | 49.05 | 5.509535735 |
| 84-2 | 50/50 | 10 | 51.92 | 48.52 | 6.54853621 |
| 84-2 | 50/50 | 11 | 53.09 | 48.89 | 7.911094368 |
| 84-2 | 50/50 | 12 | 50.82 | 46.67 | 8.166076348 |
| 84-2 | 50/50 | 13 | 51.72 | 47.25 | 8.642691415 |
| 91-2 | 50/50 | 1 | 55.16 | 51.6 | 6.453952139 |
| 91-2 | 50/50 | 2 | 54.74 | 51.56 | 5.809280234 |
| 91-2 | 50/50 | 3 | 54.32 | 51.82 | 4.602356406 |
| 91-2 | 50/50 | 4 | 56.06 | 51.43 | 8.259008205 |
| 91-2 | 50/50 | 5 | 55.29 | 51.25 | 7.306927112 |
| 91-2 | 50/50 | 6 | 55.68 | 51.5 | 7.507183908 |
| 91-2 | 50/50 | 7 | 55.01 | 51.49 | 6.398836575 |
| 91-2 | 50/50 | 8 | 54.63 | 51.5 | 5.729452682 |
| 91-2 | 50/50 | 9 | 55.11 | 51.11 | 7.258210851 |
| 91-2 | 50/50 | 10 | 54.75 | 50.89 | 7.050228311 |
| 91-2 | 50/50 | 11 | 54.61 | 50.84 | 6.903497528 |
| 91-2 | 50/50 | 12 | 54.37 | 51.08 | 6.051131138 |
| 91-3 | 50/50 | 1 | 55.12 | 51.27 | 6.984760522 |
| 91-3 | 50/50 | 2 | 54.37 | 50.98 | 6.235056097 |
| 91-3 | 50/50 | 3 | 55.27 | 51.12 | 7.508594174 |
| 91-3 | 50/50 | 4 | 55.1 | 50.92 | 7.586206897 |
| 91-3 | 50/50 | 5 | 53.67 | 50.94 | 5.086640581 |
| 91-3 | 50/50 | 6 | 53.81 | 52.06 | 3.252183609 |
| 91-3 | 50/50 | 7 | 55.08 | 51.74 | 6.063907044 |
| 91-3 | 50/50 | 8 | 55.1 | 51.72 | 6.13430127 |
| 91-3 | 50/50 | 9 | 54.39 | 51.51 | 5.295091009 |
| 91-3 | 50/50 | 10 | 55.02 | 52.18 | 5.16175936 |
| 91-3 | 50/50 | 11 | 53.76 | 51.22 | 4.724702381 |
| 91-3 | 50/50 | 12 | 53.69 | 51.28 | 4.488731607 |
| 93-2 | 50/50 | 1 | 56.45 | 53.97 | 4.393268379 |
| 93-2 | 50/50 | 2 | 56.24 | 53.69 | 4.534139403 |
| 93-2 | 50/50 | 3 | 55.31 | 53.33 | 3.579822817 |
| 93-2 | 50/50 | 4 | 56.4 | 52.98 | 6.063829787 |
| 93-2 | 50/50 | 5 | 55.6 | 53.6 | 3.597122302 |
| 93-2 | 50/50 | 6 | 55.77 | 53.81 | 3.514434284 |
| 93-2 | 50/50 | 7 | 55.49 | 53.19 | 4.144890971 |
| 93-2 | 50/50 | 8 | 55.45 | 53.27 | 3.931469793 |
| 93-2 | 50/50 | 9 | 55.03 | 53.17 | 3.379974559 |
| 93-2 | 50/50 | 10 | 55.69 | 53.83 | 3.3399174 |
| 93-2 | 50/50 | 11 | 55.44 | 53.24 | 3.968253968 |
| 99-3 | 50/50 | 1 | 54.13 | 51.88 | 4.156659893 |
| 99-3 | 50/50 | 2 | 54.52 | 52.28 | 4.108584006 |
| 99-3 | 50/50 | 3 | 53.52 | 51.87 | 3.082959641 |
| 99-3 | 50/50 | 4 | 54.05 | 52.87 | 2.183163737 |
| 99-3 | 50/50 | 5 | 55.66 | 53.74 | 3.449514912 |
| 99-3 | 50/50 | 6 | 55.98 | 53.78 | 3.929974991 |
| 99-3 | 50/50 | 7 | 56.12 | 54.04 | 3.70634355 |
| 99-3 | 50/50 | 8 | 55.51 | 53.87 | 2.954422627 |
| 99-3 | 50/50 | 9 | 55.08 | 53.44 | 2.977487291 |
| 99-3 | 50/50 | 10 | 56.16 | 52.98 | 5.662393162 |
| 99-3 | 50/50 | 11 | 56.02 | 53.37 | 4.730453409 |
| 99-3 | 50/50 | 12 | 55.83 | 53.89 | 3.474834318 |

### Example 6

### Methanol-Annealing Spun Blend Fibers

Various blend fibers were produced according to similar conditions as those described above with respect to Example 3 and were annealed with methanol to potentially increase the crystallinity of 18B protein in the fiber. Fibers were placed in a sealed container with methanol at room temperature for a duration of time sufficient for the methanol to evaporate. The methanol annealed fibers had an improve drape and handfeel after water treatment relative to fibers that were not subject to methanol vapor annealing.

### Example 7

### Rheology of Dopes Using New Celluloses and Mixing Techniques

Additional methods of mixing dopes comprising cellulose and the 18B powder described above in Example 1 (lot 153′) and other 18B powder lots that were substantially similar in purity to lot 153 were investigated. Specifically, in addition to a lab mixer substantially similar to that described in Example 2, a Ross Mixer DPM-2 mixer (“Industrial Mixer”) and a LIST KneaderReactor mixer (“Industrial Kneader”) were used to create various dopes comprising 18B Powder and cellulose dissolved in NMMO. Similarly, new types of cellulose were used to investigate the effect of the type of cellulose on the mixture. Micro-crystalline cellulose from Sigma Aldrich (“MCC”); dissolving wood pulp obtained from Georgia-Pacific (“GP-VFC”); and dissolving wood pulp from Domsjo (“Domsjo”).

For the Domsjo and GP-VFC dissolving wood pulp samples (listed in Table 7 with the DAR prefix), NMMO monohydrate (Carbosynth) was dissolved in water to form 50% aqueous solution, then mixed with ground wood pulp (Domsjo), protein powder 18B, and propyl gallate at 69° C. under nitrogen purge to form a slurry type mixture in a customized Chemglass jacketed reaction vessel equipped with PTFE coated stainless steel impeller with anchor type blades. The jacket vessel was heated to 100-110° C., nitrogen was removed, and a vacuum was applied using a Vario PC 3001 vacuum pump with pressure control to remove excess of water (step wise, from 100 torr down to 35 torr), until NMMO monohydrate was formed and a homogeneous dope was generated.

Ratios of cellulose to 18B Powder by weight % investigated included 50% cellulose by weight/50% 18B Powder by weight; 69% cellulose by weight/31% 18B Powder by weight; 67% cellulose by weight/33% 18B Powder by weight; and 75% cellulose by weight/25% 18B Powder by weight. The cellulose and 18B Powder were dissolved in NMMO. Amount of solids (cellulose and 18B Powder) by weight in each dope is listed below. The remaining % by weight was NMMO.

Phase angle and Complex Viscosity were measured as above for Experiment 2 and are tabulated below in Table 7 along with the composition of the dope.

TABLE 7
Dope Composition and Rheology

| | | | | | Dope solid | | Complex | Phase | |
|---|---|---|---|---|---|---|---|---|---|
| | | | | | content | Propyl | viscosity | angle at | |
| Dope | Cellulose | 18B | Cellulose | 18B | cellulose + 18B | gallate | at 10 Hz | 10 Hz | |
| number | type | lot no. | (wt %) | (wt %) | (wt %) | (wt %) | (Pa · s) | (°) | Mixer type |
| 107 | MCC | 153 | 70 | 30 | 20 | 0.8 | 90.21 | 62.06 | Lab mixer |
| 108 | MCC | 153 | 70 | 30 | 20 | 0.8 | 130.2 | 58.84 | Lab mixer |
| 111 | MCC | 153 | 67 | 33 | 18 | 0.8 | 80.36 | 62.61 | Lab mixer |
| 112 | MCC | 153 | 70 | 30 | 20 | 0.8 | 141.4 | 51.43 | Lab mixer |
| 118 | MCC | 153 | 70 | 30 | 20 | 0.8 | 119.9 | 59.09 | Lab mixer |
| 120 | MCC | 153 | 69 | 31 | 21 | 0.8 | 147.3 | 57.08 | Lab mixer |
| 122 | MCC | 153 | 69 | 31 | 21 | 0.8 | 137.2 | 57.54 | Lab mixer |
| 125 | MCC | 153 | 50 | 50 | 22 | 0.8 | NA | NA | Lab mixer |
| 127 | MCC | 153 | 67 | 33 | 18 | 0.8 | 61.56 | 67.64 | Lab mixer |
| 129 | MCC | 153 | 50 | 50 | 22 | 0.8 | 104.1 | 62.45 | Lab mixer |
| DAR 020 | GP-VFC | Venom 4 | 50 | 50 | 13 | 0.8 | 59.65 | 45.15 | Lab mixer |
| DAR 021 | GP-VFC | 153 | 50 | 50 | 13 | 0.8 | 45.52 | 46.25 | Lab mixer |
| DAR 024 | Domsjo | 153 | 50 | 50 | 14 | 0.8 | 78.27 | 36.9 | Lab mixer |
| DAR 025 | Domsjo | Venom 4 | 50 | 50 | 14 | 0.8 | NA | NA | Lab mixer |
| DAR 026 | Domsjo | 153 | 50 | 50 | 14 | 0.8 | 57.32 | 40.16 | Lab mixer |
| DAR 030 | Domsjo | 153 | 50 | 50 | 14 | 0.8 | 80.66 | 36.42 | Lab mixer |
| DAR 032 | Domsjo | 153 | 50 | 50 | 14 | 0.8 | 66.35 | 37.01 | Industrial |
| | | | | | | | | | Kneader |
| DAR 034 | Domsjo | 153 | 70 | 30 | 13 | 0.07 | 101.9 | 32.24 | Industrial |
| | | | | | | | | | Kneader |
| DAR 035 | Domsjo | 153 | 70 | 30 | 13 | 0.07 | 117.3 | 31.31 | Industrial |
| | | | | | | | | | Kneader |
| DAR 036 | Domsjo | 153 | 70 | 30 | 13 | 0.07 | 61.59 | 37.07 | Industrial |
| | | | | | | | | | Kneader |
| DAR 037 | Domsjo | 153 | 70 | 30 | 13 | 0.07 | 75.41 | 37.43 | Industrial |
| | | | | | | | | | Kneader |
| DAR 038 | Domsjo | 153 | 70 | 30 | 13 | 0.07 | 272.2 | 28.34 | lab mixer |
| DAR 041 | Domsjo | 153 | 70 | 30 | 13 | 0.07 | 115.9 | 32.35 | Industrial |
| | | | | | | | | | Kneader |
| DAR 043B | Domsjo | Venom 4 | 70 | 30 | 13 | 0.07 | 112.3 | 32.14 | lab mixer |
| DAR 043D | Domsjo | Venom 2 | 70 | 30 | 13 | 0.07 | 120.7 | 32.14 | lab mixer |
| DAR 045 | Domsjo | 153 | 70 | 30 | 13 | 0.07 | 163.3 | 31.3 | Industrial |
| | | | | | | | | | mixer |
| DAR 046 | Domsjo | 153 | 70 | 30 | 13 | 0.07 | 168.8 | 31.83 | Industrial |
| | | | | | | | | | mixer |

### Example 8

### Spin Conditions for New Dopes

Various spinning conditions were investigated for the dopes discussed above in Example 7. Except for the varying process variables listed below, the examples included below were produced using spinning conditions substantially similar to, or the same as, those discussed above with respect to Example 3. DAR dopes were spun using either a 10-hole or a 15-hole spinneret. As tabulated in Table 8 below, various process parameters varied across experiments, including extrusion rate, pump pressure and godet/winder rates. The bath into which the fiber was extruded varied and in some instances where the bath was a water bath, no air gap was used. As above, although the godet and winder rates varied between experiments, they were approximately the same within experiments and the fiber was not subject to additional drawing beyond the initial jet stretch. Data that was not available if marked “NA”.

As indicated below, in some experiments, a conditioner comprising an emulsion of oil in water with various other agents (Aussie 3 Minute Miracle Hair Conditioner) was used to treat the fiber.

TABLE 8
Process Parameters

| Dope- | Ext. rate | Air | Pump pressure | | Godet 1 | Godet 2 | Drying Oven |
|---|---|---|---|---|---|---|---|
| Fiber | μl/min | Gap | (psi) | Air Flow | m/min | (m/min) | (m/min) |
| 107-F1 | 300 | NA | 276 | Turbulent | 8 | 8 | 8 |
| 107-F2 | 300 | NA | 300 | Turbulent | 30 | 30 | 30.1 |
| 107-F3 | 300 | NA | NA | No | 30 | 30 | No |
| 107-F4 | 300 | NA | NA | Turbulent | 30 | 30 | 30.1 |
| 108-F1 | 300 | NA | 455 | Turbulent | 20 | 20 | 20.4 |
| 108-F2 | 300 | NA | NA | Turbulent | 20 | 20 | 20.4 |
| 108-F3 | 300 | NA | NA | Turbulent | 20 | 20 | 20.4 |
| 108-F4 | 300 | NA | NA | Turbulent | 20 | 20 | 20.4 |
| 108-F5 | 200 | NA | 370 | Turbulent | 15 | 15 | 15.1 |
| 112-F1 | 300 | NA | 458 | Turbulent | 20 | 20 | 20 |
| 112-F2 | 300 | NA | 538 | Turbulent | 20 | 20 | 20 |
| 112-F3 | 300 | NA | 488 | No | 30 | 30 | 30 |
| 112-F4 | 300 | NA | NA | No | 10 | 10 | 10 |
| 118-F1 | 450 | NA | 559 | No | 20 | 20 | 20 |
| 118-F2 | 450 | NA | NA | No | 20 | 20 | 20 |
| 118-F3 | 400 | NA | 544 | No | 20 | 20 | 20 |
| 120-F1 | 300 | 3 | 560 | No | 10 | 10 | 10 |
| 120-F2 | 600 | 3 | 864 | No | 15 | 15 | 15 |
| 120-F3 | 300 | 3 | NA | No | 20 | 20 | 20.4 |
| 122-F1 | 300 | 2 | 496 | No | 20 | 20 | 20.4 |
| 122-F2 | 300 | 2 | 496 | No | 20 | 20 | 20.4 |
| 122-F3 | 300 | 2 | 496 | No | 20 | 20 | 20.4 |
| 122-F4 | 300 | 2 | 490 | No | 18 | 18 | 18 |
| 122-F5 | 300 | 5 | 441 | No | 20 | 20 | 20 |
| 122-F6 | 300 | 3 | 403 | No | 20 | 20 | 20 |
| 122-F7 | 300 | 4 | 397 | No | 17 | 17 | 17 |
| 122-F8 | 450 | 1.5 | 568 | No | 20 | 20 | 20 |
| 122-F9 | 450 | 1.5 | 585 | No | 20 | 20 | 20 |
| 122-F10 | 450 | 1.5 | 600 | No | 22 | 22 | 22 |
| 125-F1 | 500 | NA | 1630 | No | 23 | 23 | 23 |
| 125-F2 | 500 | NA | 1725 | No | 30 | 30 | 30 |
| 125-F3 | 450 | NA | 1700 | No | 30 | 30 | 30 |
| 127-F1 | 300 | NA | 769 | No | 20 | 20 | 20 |
| 127-F2 | 300 | NA | 778 | No | 30 | 30 | 30 |
| DAR 020-F1 | 300 | 3 | 680 | No | 20 | 20 | 20 |
| DAR 020-F2 | 400 | 3 | 848 | No | 20 | 20 | 20 |
| DAR 020-F3 | 300 | 3 | 742 | No | 20 | 20 | 20 |
| DAR 020-F4 | 300 | 3 | 795 | No | 15 | 15 | 15 |
| DAR 020-F5 | 400 | 3 | 1080 | No | 20 | 20 | 20 |
| DAR 021-F1 | 400 | 3 | 694 | No | 20 | 20 | 20 |
| DAR 021-F2 | 300 | 3 | 668 | No | 20 | 20 | 20 |
| DAR 021-F3 | 300 | 3 | 722 | No | 20 | 20 | 20 |
| DAR 021-F4 | 300 | 3 | 743 | No | 15 | 15 | 15 |
| DAR 021-F5 | 400 | 3 | 925 | No | 20 | 20 | 20 |
| DAR 024-F1 | 300 | 3 | 1238 | Turbulent | 20 | 20 | 20 |
| DAR 024-F2 | 300 | 3 | 1351 | Turbulent | 20 | 20 | 20 |
| DAR 024-F3 | 400 | 3 | 1710 | Turbulent | 20 | 20 | 20 |
| DAR 024-F4 | 300 | 3 | 1523 | Turbulent | 15 | 15 | 15 |
| DAR 024-F5 | 300 | 3 | 1534 | Turbulent | 20 | 20 | 20 |
| DAR 025-F1 | 300 | 3 | 853 | Turbulent | 15 | 15 | 15 |
| DAR 025-F2 | 300 | 3 | 855 | Turbulent | 20 | 20 | 20 |
| DAR 025-F3 | 400 | 3 | 1049 | Turbulent | 20 | 20 | 20 |
| DAR 026-F1 | 300 | 3 | 687 | Turbulent | 20 | 20 | 20 |
| DAR 026-F2 | 300 | 3 | 793 | Turbulent | 20 | 20 | 20 |
| DAR 026-F3 | 300 | 3 | 803 | Turbulent | 20 | 20 | 20 |
| DAR 026-F4 | 300 | 3 | 850 | Turbulent | 20 | 20 | 20 |
| DAR 026-F5 | 300 | 3 | 881 | Turbulent | 20 | 20 | 20 |
| DAR 026-F6 | 300 | 3 | 914 | Turbulent | 20 | 20 | 20 |
| DAR 026-F7 | 300 | 3 | 965 | Turbulent | 20 | 20 | 20 |
| DAR 026-F8 | 300 | 3 | 991 | Turbulent | 20 | 20 | 20 |
| DAR 026-F9 | 300 | 3 | 1176 | Turbulent | 20 | 20 | 20 |
| DAR 026-F10 | 300 | 3 | 1328 | Turbulent | 20 | 20 | 20 |
| DAR 026-F11 | 300 | 3 | 1575 | Turbulent | 20 | 20 | 20 |
| DAR 026-F12 | 300 | 3 | 1198 | Turbulent | 20 | 20 | 20 |
| DAR 026-F13 | 300 | 3 | 943 | Turbulent | 20 | 20 | 20 |
| DAR 026-F14 | 300 | 3 | 941 | Turbulent | 20 | 20 | 20 |
| DAR 026-F15 | 300 | 3 | 970 | Turbulent | 20 | 20 | 20 |
| DAR 030 - F1 | 300 | 3 | 695 | Turbulent | 20 | 20 | 20 |
| DAR 030 - F2 | 300 | 3 | 732 | Turbulent | 20 | 20 | 20 |
| DAR 030 - F3 | 300 | 3 | 747 | Turbulent | 20 | 20 | 20 |
| DAR 030 - F4 | 300 | 3 | NA | Turbulent | 20 | 20 | 20 |
| DAR 030 - F5 | 300 | 3 | NA | Turbulent | 20 | 20 | 20 |
| DAR 030 - F6 | 300 | 3 | 742 | Turbulent | 20 | 20 | 20 |
| DAR 030 - F7 | 300 | 3 | 767 | Turbulent | 20 | 20 | 20 |
| DAR 030 - F8 | 300 | 3 | 822 | Turbulent | 20 | 20 | 20 |
| DAR 030 - F9 | 300 | 3 | 834 | Turbulent | 20 | 20 | 20 |
| DAR 030 - F10 | 300 | 3 | 843 | Turbulent | 20 | 20 | 20 |
| DAR 030 - F11 | 300 | 3 | 887 | Turbulent | 20 | 20 | 20 |
| DAR 030 - F12 | 300 | 3 | 909 | Turbulent | 20 | 20 | 20 |
| DAR 030 - F13 | 300 | 3 | 930 | Turbulent | 20 | 20 | 20 |
| DAR 030 - F14 | 300 | 3 | 963 | Turbulent | 20 | 20 | 20 |
| DAR 030 - F15 | 300 | 3 | 999 | Turbulent | 20 | 20 | 20 |
| DAR 030 - F16 | 300 | 3 | 1059 | Turbulent | 20 | 20 | 20 |
| DAR 030 - F17 | 300 | 3 | 1120 | Turbulent | 20 | 20 | 20 |
| DAR 032-F1 | 300 | 3 | 670 | Turbulent | 20 | 20 | 20 |
| DAR 032-F2 | 300 | 3 | 677 | Turbulent | 15 | 15 | 15 |
| DAR 032-F3 | 400 | 3 | 687 | Turbulent | 20 | 20 | 20 |
| DAR 032-F4 | 300 | 3 | 687 | Turbulent | 20 | 20 | 20 |
| DAR 032-F5 | 300 | 3 | 687 | Turbulent | 20 | 20 | 20 |
| DAR 034-F1 | 300 | 3 | 984 | No | 20 | 20 | 20 |
| DAR 034-F2 | 300 | 3 | 1004 | No | 20 | 20 | 20 |
| DAR 034-F3 | 300 | 3 | 1015 | No | 15 | 15 | 15 |
| DAR 034-F4 | 400 | 3 | 1168 | No | 20 | 20 | 20 |
| DAR 034-F5 | 300 | 3 | 1050 | No | 20 | 20 | 20 |
| DAR 034-F6 | 300 | 3 | 1080 | No | 20 | 20 | 20 |
| DAR 034-F7 | 300 | 3 | 1096 | No | 25 | 25 | 25 |
| DAR 035-F1 | 500 | 3 | 1841 | No | 20 | 20 | 20 |
| DAR 035-F2 | 500 | 3 | 1844 | No | 15 | 15 | 15 |
| DAR 035-F3 | 500 | 3 | 1850 | No | 20 | 19.5 | 19.5 |
| DAR 035-F4 | 500 | 3 | 1853 | No | 20 | 20 | 20 |
| DAR 035-F5 | 500 | 3 | 1864 | No | 20 | 20 | 20 |
| DAR 035-F6 | 300 | 3 | 1690 | No | 20 | 19.5 | 19.5 |
| DAR 035-F7 | 300 | 3 | 1690 | No | 20 | 20 | 20 |
| DAR 035-F8 | 300 | 3 | 1688 | No | 20 | 20 | 20 |
| DAR 036-F1 | 300 | 3 | 998 | No | 20 | 20 | 20 |
| DAR 036-F2 | 300 | 3 | 999 | No | 15 | 15 | 15 |
| DAR 036-F3 | 300 | 3 | 1001 | No | 25 | 25 | 25 |
| DAR 036-F4 | 300 | 3 | 1003 | No | 20 | 20 | 20 |
| DAR 036-F5 | 600 | 3 | 1412 | No | 40 | 40 | 40 |
| DAR 036-F6 | 900 | 3 | 1720 | No | 60 | 60 | 60 |
| DAR 037-F1 | 300 | 3 | 981 | No | 20 | 20 | 20 |
| DAR 037-F2 | 300 | 8 | 1001 | No | 20 | 20 | 20 |
| DAR 037-F3 | 1500 | 5 | NA | No | 20 | 20 | 20 |
| DAR 037-F4 | 3000 | 5 | NA | No | 40 | 40 | 40 |
| DAR 037-F5 | 3000 | 1 | NA | No | 40 | 40 | 40 |
| DAR 038-F1 | 300 | 3 | 863 | No | 20 | 20 | 20 |
| DAR 038-F2 | 300 | 3 | NA | No | 20 | 20 | 20 |
| DAR 038-F3 | 1750 | 1 | NA | No | 20 | 20 | 20 |
| DAR 041-F1 | 300 | 3 | 1579 | No | 20 | 20 | 20 |
| DAR 041-F2 | 300 | 3 | 1623 | No | 20 | 20 | 20 |
| DAR 043B-F1 | 300 | 3 | 1115 | No | 20 | 20 | 20 |
| DAR 043B-F2 | 300 | 3 | 1123 | No | 20 | 20 | 20 |
| DAR 043D-F5 | 300 | 3 | 1213 | No | 20 | 20 | 20 |
| DAR 043D-F6 | 300 | 3 | 1199 | No | 20 | 20 | 20 |
| DAR 046-SP37 | 300 | 3 | 1098 | No | 20 | 20 | 20 |
| DAR 046-SP44 | 300 | 3 | 1096 | No | 20 | 20 | 20 |

| Dope- | Godet 3 | Winder | | | | Fiber finish | | Jet |
|---|---|---|---|---|---|---|---|---|
| Fiber | (m/min) | (m/min) | Bath1 | Drip 1 | Bath2 | (conditioner) | Drip2 | Stretch |
| 107-F1 | 8.1 | 8.1 | Water | Water | Ethanol | No | No | 3.38 |
| 107-F2 | 30.5 | 30.7 | Water | Water | Ethanol | No | No | 12.66 |
| 107-F3 | 30.5 | 30.7 | Water | Water | Ethanol | No | No | 12.66 |
| 107-F4 | 30.5 | 30.7 | Water | Water | Ethanol | No | No | 12.66 |
| 108-F1 | 20.5 | 21 | Water | Water | Ethanol | No | No | 8.44 |
| 108-F2 | 20.5 | 21 | Water | Water | Ethanol | No | No | 8.44 |
| 108-F3 | 20.5 | 21 | Water | Water | Ethanol | No | No | 8.44 |
| 108-F4 | 20.3 | 20.8 | Water | Water | Ethanol | No | No | 8.44 |
| 108-F5 | 15.3 | 15.4 | Water | Water | Ethanol | No | No | 9.49 |
| 112-F1 | 20.3 | 20.3 | Water | Water | Ethanol | No | No | 8.44 |
| 112-F2 | 20.3 | 20.6 | Water | Water | Ethanol | Yes | No | 8.44 |
| 112-F3 | 30.3 | 30.7 | Water | Water | Ethanol | No | No | 12.66 |
| 112-F4 | 10.1 | 10.3 | Water | Water | Ethanol | Yes | No | 4.22 |
| 118-F1 | 20.4 | 20.8 | Water | Water | Water | Yes | No | 8.44 |
| 118-F2 | 20.4 | 20.8 | Water | Water | Water | Yes | No | 8.44 |
| 118-F3 | 20.4 | 20.8 | Water | Water | Water | Yes | No | 6.33 |
| 120-F1 | 10.5 | 10.8 | Water | Water | Water | Yes | No | 4.22 |
| 120-F2 | 15.7 | 16.2 | Water | Water | Water | Yes | No | 3.16 |
| 120-F3 | 20.4 | 20.7 | Water | Ethanol | Water | Yes | No | 8.44 |
| 122-F1 | 20.4 | 20.7 | Water | Ethanol | Water | Yes | Ethanol | 8.44 |
| 122-F2 | 20.4 | 20.7 | Water | Ethanol | Water | Yes | Ethanol | 8.44 |
| 122-F3 | 20.4 | 20.7 | Water | Ethanol | Water | Yes | Ethanol | 8.44 |
| 122-F4 | 18 | 18.3 | Water | Ethanol | Water | Yes | Ethanol | 7.59 |
| 122-F5 | 20 | 20.8 | Water | Ethanol | Water | Yes | Ethanol | 8.44 |
| 122-F6 | 20 | 20.8 | Water | Ethanol | Water | Yes | Ethanol | 8.44 |
| 122-F7 | 17 | 17.4 | Water | Ethanol | Water | Yes | Ethanol | 7.17 |
| 122-F8 | 20 | 20.5 | 90/10 | ethanol | Water | Yes | Ethanol | 8.44 |
| | | | Ethanol/ | | | | | |
| | | | water | | | | | |
| 122-F9 | 20 | 20.5 | 90/10 | ethanol | Water | Yes | Ethanol | 8.44 |
| | | | Ethanol/ | | | | | |
| | | | water | | | | | |
| 122-F10 | 22 | 22.5 | 90/10 | ethanol | Water | Yes | Ethanol | 9.28 |
| | | | Ethanol/ | | | | | |
| | | | water | | | | | |
| 125-F1 | 23 | 23.5 | Water | Ethanol | Water | Yes | Ethanol | 8.73 |
| 125-F2 | 30 | 30.5 | Water | Ethanol | Water | Yes | Ethanol | 11.39 |
| 125-F3 | 30 | 30.5 | Water | Ethanol | Water | No | Ethanol | 12.66 |
| 127-F1 | 20 | 20.9 | Water | Ethanol | Water | Yes | Ethanol | 8.44 |
| 127-F2 | 30 | 30.9 | Water | Ethanol | Water | Yes | Ethanol | 12.66 |
| DAR 020-F1 | 20.2 | 20.4 | Water | Water | Water | Yes | No | 8.44 |
| DAR 020-F2 | 20.2 | 20.4 | Water | Water | Water | Yes | No | 6.33 |
| DAR 020-F3 | 20.2 | 20.4 | Water | Water | Water | Yes | No | 8.44 |
| DAR 020-F4 | 15.2 | 15.3 | Water | Water | Water | Yes | No | 6.33 |
| DAR 020-F5 | 20.2 | 20.4 | Water | Water | Water | Yes | No | 6.33 |
| DAR 021-F1 | 20.2 | 20.4 | Water | Water | Water | Yes | No | 6.33 |
| DAR 021-F2 | 20.2 | 20.4 | Water | Water | Water | Yes | No | 8.44 |
| DAR 021-F3 | 20.2 | 20.4 | Water | Water | Water | Yes | No | 8.44 |
| DAR 021-F4 | 15.1 | 15.2 | Water | Water | Water | Yes | No | 6.33 |
| DAR 021-F5 | 20.2 | 20.4 | Water | Water | Water | Yes | No | 6.33 |
| DAR 024-F1 | 20.2 | 20.4 | Water | Water | Water | Yes | No | 8.44 |
| DAR 024-F2 | 20.2 | 20.4 | Water | Water | Water | Yes | No | 8.44 |
| DAR 024-F3 | 20.2 | 20.4 | Water | Water | Water | Yes | No | 6.33 |
| DAR 024-F4 | 15.2 | 15.3 | Water | Water | Water | Yes | No | 6.33 |
| DAR 024-F5 | 20.2 | 20.4 | Water | Water | Water | Yes | No | 8.44 |
| DAR 025-F1 | 15.2 | 15.3 | Water | Water | Water | Yes | No | 6.33 |
| DAR 025-F2 | 20.2 | 20.4 | Water | Water | Water | Yes | No | 8.44 |
| DAR 025-F3 | 20.2 | 20.4 | Water | Water | Water | Yes | No | 6.33 |
| DAR 026-F1 | 20.2 | 20.4 | Water | Water | Water | Yes | No | 8.44 |
| DAR 026-F2 | 20.2 | 20.4 | Water | Water | Water | Yes | No | 8.44 |
| DAR 026-F3 | 20.2 | 20.4 | Water | Water | Water | Yes | No | 8.44 |
| DAR 026-F4 | 20.2 | 20.4 | Water | Water | Water | Yes | No | 8.44 |
| DAR 026-F5 | 20.2 | 20.4 | Water | Water | Water | Yes | No | 8.44 |
| DAR 026-F6 | 20.2 | 20.4 | Water | Water | Water | Yes | No | 8.44 |
| DAR 026-F7 | 20.2 | 20.4 | Water | Water | Water | Yes | No | 8.44 |
| DAR 026-F8 | 20.2 | 20.4 | Water | Water | Water | Yes | No | 8.44 |
| DAR 026-F9 | 20.2 | 20.4 | Water | Water | Water | Yes | No | 8.44 |
| DAR 026-F10 | 20.2 | 20.4 | Water | Water | Water | Yes | No | 8.44 |
| DAR 026-F11 | 20.2 | 20.4 | Water | Water | Water | Yes | No | 8.44 |
| DAR 026-F12 | 20.2 | 20.4 | Water | Water | Water | Yes | No | 8.44 |
| DAR 026-F13 | 20.2 | 20.4 | Water | Water | Water | Yes | No | 8.44 |
| DAR 026-F14 | 20.2 | 20.4 | Water | Water | Water | Yes | No | 8.44 |
| DAR 026-F15 | 20.2 | 20.4 | Water | Water | Water | Yes | No | 8.44 |
| DAR 030 - F1 | 20.2 | 20.4 | Water | Water | Water | Yes | No | 8.44 |
| DAR 030 - F2 | 20.2 | 20.4 | Water | Water | Water | Yes | No | 8.44 |
| DAR 030 - F3 | 20.2 | 20.4 | Water | Water | Water | Yes | No | 8.44 |
| DAR 030 - F4 | 20.2 | 20.4 | Water | Water | Water | Yes | No | 8.44 |
| DAR 030 - F5 | 20.2 | 20.4 | Water | Water | Water | Yes | No | 8.44 |
| DAR 030 - F6 | 20.2 | 20.4 | Water | Water | Water | Yes | No | 8.44 |
| DAR 030 - F7 | 20.2 | 20.4 | Water | Water | Water | Yes | No | 8.44 |
| DAR 030 - F8 | 20.2 | 20.4 | Water | Water | Water | Yes | No | 8.44 |
| DAR 030 - F9 | 20.2 | 20.4 | Water | Water | Water | Yes | No | 8.44 |
| DAR 030 - F10 | 20.2 | 20.4 | Water | Water | Water | Yes | No | 8.44 |
| DAR 030 - F11 | 20.2 | 20.4 | Water | Water | Water | Yes | No | 8.44 |
| DAR 030 - F12 | 20.2 | 20.4 | Water | Water | Water | Yes | No | 8.44 |
| DAR 030 - F13 | 20.2 | 20.4 | Water | Water | Water | Yes | No | 8.44 |
| DAR 030 - F14 | 20.2 | 20.4 | Water | Water | Water | Yes | No | 8.44 |
| DAR 030 - F15 | 20.2 | 20.4 | Water | Water | Water | Yes | No | 8.44 |
| DAR 030 - F16 | 20.2 | 20.4 | Water | Water | Water | Yes | No | 8.44 |
| DAR 030 - F17 | 20.2 | 20.4 | Water | Water | Water | Yes | No | 8.44 |
| DAR 032-F1 | 20.2 | 20.4 | Water | Water | Water | Yes | No | 8.44 |
| DAR 032-F2 | 15.1 | 15.3 | Water | Water | Water | Yes | No | 6.33 |
| DAR 032-F3 | 20.2 | 20.4 | Water | Water | Water | Yes | No | 6.33 |
| DAR 032-F4 | 20.2 | 20.4 | Water | Water | Water | Yes | No | 8.44 |
| DAR 032-F5 | 20.2 | 20.4 | Water | Water | Water | Yes | No | 8.44 |
| DAR 034-F1 | 20.2 | 20.4 | Water | Water | Water | Yes | No | 8.44 |
| DAR 034-F2 | 20.2 | 20.4 | Water | Water | Water | Yes | No | 8.44 |
| DAR 034-F3 | 15.2 | 15.3 | Water | Water | Water | Yes | No | 6.33 |
| DAR 034-F4 | 20.2 | 20.4 | Water | Water | Water | Yes | No | 6.33 |
| DAR 034-F5 | 20.2 | 20.4 | Water | Water | Water | Yes | No | 8.44 |
| DAR 034-F6 | 20.2 | 20.4 | Water | Water | Water | Yes | No | 8.44 |
| DAR 034-F7 | 25.3 | 25.5 | Water | Water | Water | Yes | No | 10.55 |
| DAR 035-F1 | 20.2 | 20.4 | Water | Water | Water | Yes | No | 5.06 |
| DAR 035-F2 | 15.2 | 15.3 | Water | Water | Water | Yes | No | 3.80 |
| DAR 035-F3 | 19.7 | 19.8 | Water | Water | Water | Yes | No | 5.06 |
| DAR 035-F4 | 20.2 | 20.4 | Water | Water | Water | Yes | No | 5.06 |
| DAR 035-F5 | 20.2 | 20.4 | Water | Water | Water | Yes | No | 5.06 |
| DAR 035-F6 | 19.7 | 19.8 | Water | Water | Water | Yes | No | 8.44 |
| DAR 035-F7 | 20.2 | 20.4 | Water | Water | Water | Yes | No | 8.44 |
| DAR 035-F8 | 20.2 | 20.4 | Water | Water | Water | Yes | No | 8.44 |
| DAR 036-F1 | 20.2 | 20.4 | Water | Water | Water | Yes | No | 8.44 |
| DAR 036-F2 | 15.2 | 15.3 | Water | Water | Water | Yes | No | 6.33 |
| DAR 036-F3 | 25.3 | 25.5 | Water | Water | Water | Yes | No | 10.55 |
| DAR 036-F4 | 20.2 | 20.4 | Water | Water | Water | Yes | No | 8.44 |
| DAR 036-F5 | 40.4 | 40.8 | Water | Water | Water | Yes | No | 8.44 |
| DAR 036-F6 | 60.6 | 61.2 | Water | Water | Water | Yes | No | 8.44 |
| DAR 037-F1 | 20.2 | 20.4 | Water | Water | Water | Yes | No | 8.44 |
| DAR 037-F2 | 20.2 | 20.4 | Water | Water | Water | Yes | No | 8.44 |
| DAR 037-F3 | 20.2 | 20.4 | Water | Water | Water | Yes | No | 8.44 |
| DAR 037-F4 | 40.4 | 40.8 | Water | Water | Water | Yes | No | 8.44 |
| DAR 037-F5 | 40.4 | 40.8 | Water | Water | Water | Yes | No | 8.44 |
| DAR 038-F1 | 20.2 | 20.4 | Water | Water | Water | Yes | No | 8.44 |
| DAR 038-F2 | 20.2 | 20.4 | Water | Water | Water | Yes | No | 8.44 |
| DAR 038-F3 | 20.2 | 20.4 | Water | Water | Water | Yes | No | 7.23 |
| DAR 041-F1 | 20.2 | 20.4 | Water | Water | Water | Yes | No | 8.44 |
| DAR 041-F2 | 20.2 | 20.4 | Water | Water | Water | Yes | No | 8.44 |
| DAR 043B-F1 | 20.2 | 20.4 | Water | Water | Water | Yes | No | 8.44 |
| DAR 043B-F2 | 20.2 | 20.4 | Water | Water | Water | Yes | No | 8.44 |
| DAR 043D-F5 | 20.2 | 20.4 | Water | Water | Water | Yes | No | 8.44 |
| DAR 043D-F6 | 20.2 | 20.4 | Water | Water | Water | Yes | No | 8.44 |
| DAR 046-SP37 | 20.2 | 20.4 | Water | Water | Water | Yes | No | 8.44 |
| DAR 046-SP44 | 20.2 | 20.4 | Water | Water | Water | Yes | No | 8.44 |

### Example 9

### Mechanical Properties of New Fibers

Mechanical properties of fiber from the Experiment 8 were measured using a FAVIMAT model tensile test equipment model Favimat+ and Robot2 using either a single filament or a tow of at least 2 filaments. Linear density was measured in accordance with ASTM D1577. Tensile properties were measured in accordance with ASTM D3822-14. Each of the properties were measured a number of times as listed tabulated below (“# of samples”) and the mean was calculated. Properties for the various fibers are tabulated below.

TABLE 8
Fiber Mechanical Properties

| | | | Mean | Mean | Mean | Mean | Mean | | | |
|---|---|---|---|---|---|---|---|---|---|---|
| | | Single | Linear | Elongation | Elongation | Maximum | Work | Mean | Initial. | |
| Dope- | Cellulose/ | filament | Density | (Fmax) | At Break | Force | (break) | Tenacity | Modulus | Number of |
| Fiber | 18B ratio | or tow | (dtex) | (%) | (%) | (cN) | (cn*cm) | (cN/tex) | (cN/tex) | samples |
| 107_F1 | 70/30 | single | 9.59 | 26.87 | 27.13 | 11.56 | 4.81 | 12.08 | 587.37 | 15 |
| 107_F2 | 70/30 | single | 2.73 | 5.99 | 6.21 | 5.07 | 0.49 | 18.76 | 1244.98 | 15 |
| 107_F3 | 70/30 | single | 2.84 | 7.86 | 8.09 | 5.41 | 0.65 | 19.06 | 1153.94 | 15 |
| 107_F4 | 70/30 | single | 2.68 | 5.29 | 5.51 | 5.07 | 0.42 | 18.98 | 1254.45 | 15 |
| 108_F1 | 70/30 | single | 3.7 | 7.52 | 7.75 | 7.45 | 0.87 | 20.33 | 1154.19 | 15 |
| 108_F2 | 70/30 | single | 3.89 | 8.03 | 8.26 | 7.56 | 0.98 | 19.56 | 1139.96 | 15 |
| 108_F3 | 70/30 | single | 3.85 | 6.59 | 6.81 | 7.38 | 0.78 | 19.2 | 1215.62 | 14 |
| 108_F4 | 70/30 | single | 3.06 | 7.84 | 8.06 | 5.71 | 0.73 | 18.74 | 1152.62 | 15 |
| 108_F5 | 70/30 | single | 3.5 | 9.72 | 9.96 | 5.88 | 0.9 | 17.14 | 1027.27 | 15 |
| 112_F1 | 70/30 | single | 3.98 | 9.4 | 9.64 | 6.45 | 0.97 | 16.22 | 1022 | 15 |
| 112_F2 | 70/30 | single | 3.76 | 6.26 | 6.49 | 6.68 | 0.66 | 17.78 | 1160.03 | 15 |
| 112_F3 | 70/30 | single | 2.8 | 4.55 | 4.78 | 5.24 | 0.36 | 18.78 | 1248.36 | 14 |
| 112_F4 | 70/30 | single | 6.17 | 11.36 | 11.6 | 8.33 | 1.47 | 13.51 | 786.3 | 15 |
| 118_F1 | 70/30 | single | 3.68 | 5.98 | 6.2 | 7.66 | 0.69 | 20.88 | 1305.45 | 25 |
| 118_F2 | 70/30 | single | 3.61 | 4.56 | 4.79 | 7.4 | 0.51 | 20.75 | 1342.82 | 22 |
| 118_F3 | 70/30 | single | 4.65 | 5.83 | 6.05 | 8.91 | 0.77 | 19.33 | 1202.11 | 25 |
| 120_F1 | 69/31 | single | 8.24 | 7.09 | 7.32 | 12.88 | 1.45 | 15.7 | 975.19 | 25 |
| 120_F2 | 69/31 | single | 10.53 | 9.21 | 9.43 | 16.68 | 2.45 | 15.85 | 926.49 | 24 |
| 120_F3 | 69/31 | single | 4.18 | 6.27 | 6.49 | 6.94 | 0.67 | 16.6 | 1043.58 | 24 |
| 122_F1 | 69/31 | tow (10) | 34.59 | 5.74 | 6.24 | 79.68 | 6.54 | 23.04 | 1266.28 | 13 |
| 122_F5 | 69/31 | tow (10) | 22.24 | 5.76 | 6.62 | 40.93 | 3.69 | 18.46 | 1113.76 | 15 |
| 122_F7 | 69/31 | single | 5.2 | 6.69 | 6.92 | 7.34 | 0.8 | 14.4 | 947.2 | 25 |
| 122_F8 | 69/31 | single | 4.07 | 5.6 | 5.82 | 6.83 | 0.58 | 16.97 | 1118.43 | 25 |
| 122_F10 | 69/31 | single | 3.81 | 3.99 | 4.21 | 6.21 | 0.37 | 16.41 | 1148.36 | 24 |
| 125_F1 | 68/32 | single | 6.45 | 5.17 | 5.4 | 9.74 | 0.77 | 15.09 | 1027.06 | 21 |
| 125_F2 | 68/32 | single | 4.48 | 5.06 | 5.29 | 7.61 | 0.58 | 17.01 | 1141.34 | 24 |
| 125_F3 | 68/32 | tow (15) | 32.41 | 7.19 | 7.72 | 77.5 | 8.15 | 24.02 | 1316.36 | 24 |
| 127_F1 | 67/33 | tow (10) | 21.48 | 4.83 | 5.32 | 44.91 | 3.26 | 20.95 | 1275.76 | 15 |
| 127_F2 | 67/33 | tow (10) | 18.17 | 5.28 | 6.71 | 37.35 | 2.93 | 20.8 | 1248.61 | 15 |
| DAR020_F1 | 50/50 | single | 2.38 | 3.86 | 4.07 | 5.95 | 0.33 | 25.25 | 1528.89 | 19 |
| DAR020_F2 | 50/50 | single | 2.91 | 3.83 | 4.04 | 6.41 | 0.35 | 22.22 | 1380.28 | 22 |
| DAR020_F3 | 50/50 | single | 2.16 | 3.78 | 4.01 | 5.32 | 0.29 | 24.87 | 1533.37 | 22 |
| DAR020_F4 | 50/50 | single | 3.05 | 4.6 | 4.82 | 6.85 | 0.47 | 22.63 | 1369.67 | 25 |
| DAR020_F5 | 50/50 | single | 2.88 | 4.18 | 4.4 | 6.66 | 0.41 | 23.13 | 1438.84 | 24 |
| DAR021_F1 | 50/50 | single | 2.59 | 3.75 | 3.97 | 6.46 | 0.34 | 25.02 | 1560.37 | 21 |
| DAR021_F2 | 50/50 | single | 2.07 | 3.22 | 3.43 | 5.2 | 0.23 | 25.33 | 1602.63 | 22 |
| DAR021_F3 | 50/50 | single | 1.78 | 3.31 | 3.54 | 4.73 | 0.22 | 26.7 | 1671.82 | 21 |
| DAR021_F4 | 50/50 | single | 2.43 | 4.15 | 4.39 | 5.91 | 0.37 | 24.42 | 1556.48 | 24 |
| DAR021_F5 | 50/50 | single | 2.86 | 3.96 | 4.19 | 6.53 | 0.37 | 22.98 | 1436.78 | 23 |
| DAR024_F4 | 50/50 | single | 2.84 | 4.82 | 5.04 | 8.49 | 0.57 | 29.89 | 1677.8 | 23 |
| DAR024_F5 | 50/50 | single | 2.09 | 4.85 | 5.07 | 6.63 | 0.44 | 31.83 | 1725.45 | 25 |
| DAR025_F1 | 50/50 | single | 2.78 | 5.34 | 5.56 | 8.64 | 0.63 | 31.18 | 1652.45 | 24 |
| DAR025_F2 | 50/50 | single | 2.41 | 6.61 | 6.83 | 6.91 | 0.61 | 28.79 | 1454.85 | 24 |
| DAR026_F1 | 50/50 | single | 2.12 | 3 | 3.23 | 6.41 | 0.24 | 30.52 | 1796.58 | 23 |
| DAR026_F5 | 50/50 | single | 2.14 | 3.11 | 3.32 | 6.01 | 0.25 | 28.13 | 1719.28 | 23 |
| DAR026_F10 | 50/50 | single | 2.23 | 3.46 | 3.67 | 6.52 | 0.3 | 29.3 | 1769.72 | 22 |
| DAR026_F15 | 50/50 | single | 2.45 | 3.66 | 3.87 | 7.2 | 0.36 | 29.53 | 1720.4 | 19 |
| DAR030_F1 | 50/50 | single | 2.35 | 5.56 | 5.79 | 7.36 | 0.58 | 31.33 | 1654.97 | 22 |
| DAR030_F7 | 50/50 | single | 2.25 | 4.87 | 5.09 | 6.9 | 0.47 | 30.86 | 1670.85 | 23 |
| DAR030_F10 | 50/50 | single | 2.3 | 4.81 | 5.03 | 6.99 | 0.47 | 30.34 | 1686.39 | 23 |
| DAR030_F17 | 50/50 | single | 2.14 | 4.59 | 4.81 | 6.69 | 0.43 | 31.32 | 1737.54 | 21 |
| DAR032_F1 | 50/50 | single | 2.25 | 5.52 | 5.74 | 6.7 | 0.52 | 29.95 | 1647.42 | 25 |
| DAR032_F2 | 50/50 | single | 2.75 | 6 | 6.22 | 8.26 | 0.68 | 30.14 | 1597.78 | 25 |
| DAR032_F5 | 50/50 | single | 2.12 | 5.3 | 5.51 | 6.78 | 0.49 | 31.94 | 1659.07 | 25 |
| DAR034_F2 | 70/30 | single | 1.64 | 4.82 | 5.03 | 6.73 | 0.45 | 41.8 | 2244.75 | 19 |
| DAR034_F3 | 70/30 | single | 2.4 | 6.11 | 6.32 | 9.54 | 0.84 | 40 | 2078.38 | 24 |
| DAR034_F4 | 70/30 | single | 2.57 | 6.55 | 6.77 | 9.61 | 0.86 | 37.63 | 1885.07 | 25 |
| DAR034_F5 | 70/30 | single | 2.02 | 5.78 | 6 | 8.03 | 0.64 | 39.98 | 1970.81 | 24 |
| DAR034_F7 | 70/30 | single | 1.53 | 5.53 | 5.76 | 6.47 | 0.49 | 42.28 | 2104.82 | 21 |
| DAR035_F6 | 70/30 | single | 2.15 | 5.14 | 5.37 | 8.26 | 0.62 | 38.58 | 2225.53 | 23 |
| DAR035_F7 | 70/30 | single | 2.2 | 4.28 | 4.5 | 8.51 | 0.51 | 38.72 | 2197.16 | 24 |
| DAR035_F8 | 70/30 | single | 2.24 | 5.17 | 5.38 | 8.35 | 0.61 | 37.49 | 2049.98 | 24 |
| DAR036_F1 | 70/30 | single | 2.12 | 5.1 | 5.33 | 9.31 | 0.67 | 43.96 | 2258.61 | 23 |
| DAR036_F2 | 70/30 | single | 2.8 | 4.83 | 5.06 | 11.74 | 0.82 | 42.2 | 2249.45 | 25 |
| DAR036_F3 | 70/30 | single | 1.59 | 4.55 | 4.77 | 7.24 | 0.46 | 45.7 | 2349.11 | 23 |
| DAR036_F5 | 70/30 | single | 2.06 | 4.43 | 4.65 | 8.77 | 0.52 | 42.98 | 2115.89 | 16 |
| DAR036_F6 | 70/30 | single | 2.1 | 3.78 | 3.99 | 9.47 | 0.46 | 45.21 | 2355.16 | 19 |
| DAR037_F1 | 70/30 | single | 1.96 | 4.9 | 5.12 | 7.59 | 0.52 | 38.71 | 2040.97 | 20 |
| DAR037_F2 | 70/30 | single | 2 | 4.61 | 4.82 | 8.14 | 0.51 | 41.07 | 2124.88 | 15 |
| DAR037_F3 | 70/30 | single | 1.76 | 5.48 | 5.7 | 6.29 | 0.49 | 35.99 | 1903.75 | 20 |
| DAR037_F5 | 70/30 | single | 2.01 | 3.8 | 4.03 | 7.32 | 0.37 | 36.41 | 2086.43 | 14 |
| DAR038_F1 | 70/30 | single | 2.03 | 4.73 | 4.95 | 7.92 | 0.51 | 39.18 | 2048.65 | 15 |
| DAR038_F2 | 70/30 | single | 2.33 | 5 | 5.23 | 8.82 | 0.6 | 37.95 | 2021.43 | 20 |
| DAR038_F3 | 70/30 | single | 2.46 | 4.17 | 4.39 | 8.45 | 0.48 | 34.52 | 1967.09 | 16 |
| DAR040_F1 | 70/30 | single | 1.94 | 4.72 | 4.94 | 7.4 | 0.5 | 38.39 | 2110.5 | 25 |
| DAR040_F2 | 70/30 | single | 2.21 | 4.93 | 5.16 | 8.82 | 0.61 | 40.01 | 2095.05 | 24 |
| DAR041_F1 | 70/30 | single | 2.65 | 4.67 | 4.89 | 10.2 | 0.66 | 38.63 | 2100.71 | 24 |
| DAR041_F2 | 70/30 | single | 2.37 | 3.83 | 4.04 | 9.54 | 0.48 | 40.34 | 2157.6 | 22 |
| DAR043B_F2 | 70/30 | single | 2.32 | 5.2 | 5.43 | 9.52 | 0.68 | 41.49 | 2106.45 | 24 |
| DAR046_F-SP4 | 70/30 | single | 2.08 | 5.31 | 5.55 | 8.28 | 0.6 | 41.58 | 2229.22 | 24 |

### Example 10

### Shrinkage of New Fibers

For select new fibers made according to the conditions in Example 8, shrinkage was measured as described above in Example 5. Results are included below as Table 9.

TABLE 9
Shrinkage of Cellulose-Silk Blends

| Dope- | Cellulose/ | | Initial length | Final length | Shrinkage |
|---|---|---|---|---|---|
| Fiber | 18B ratio | Sample | (mm) | (mm) | (%) |
| 107_F4 | 70/30 | 1 | 55.86 | 53.71 | 3.849 |
| 107_F4 | 70/30 | 2 | 55.32 | 53.46 | 3.362 |
| 107_F4 | 70/30 | 3 | 55.07 | 53.82 | 2.270 |
| 107_F4 | 70/30 | 4 | 55.17 | 53.15 | 3.661 |
| 107_F4 | 70/30 | 5 | 54.46 | 52.56 | 3.489 |
| 107_F4 | 70/30 | 6 | 55.55 | 53.48 | 3.726 |
| 107_F4 | 70/30 | 7 | 55.84 | 53.75 | 3.743 |
| 107_F4 | 70/30 | 8 | 55.11 | 53.9 | 2.196 |
| 107_F4 | 70/30 | 9 | 55.47 | 54.18 | 2.326 |
| 107_F4 | 70/30 | 10 | 55.29 | 53.89 | 2.532 |
| 107_F4 | 70/30 | 11 | 54.91 | 52.47 | 4.444 |
| 107_F4 | 70/30 | 12 | 55.63 | 53.72 | 3.433 |
| 118_F1 | 70/30 | 1 | 56.04 | 54.18 | 3.319 |
| 118_F1 | 70/30 | 2 | 55.48 | 53.74 | 3.136 |
| 118_F1 | 70/30 | 3 | 55.34 | 53.01 | 4.210 |
| 118_F1 | 70/30 | 4 | 56.38 | 54.46 | 3.405 |
| 118_F1 | 70/30 | 5 | 55.84 | 54.12 | 3.080 |
| 118_F1 | 70/30 | 6 | 55.59 | 53.26 | 4.191 |
| 118_F1 | 70/30 | 7 | 57.64 | 56.04 | 2.776 |
| 118_F1 | 70/30 | 8 | 56.33 | 54.32 | 3.568 |
| 118_F1 | 70/30 | 9 | 55.31 | 53.34 | 3.562 |
| 118_F1 | 70/30 | 10 | 55.84 | 53.84 | 3.582 |
| 118_F1 | 70/30 | 11 | 56.04 | 52.98 | 5.460 |
| 118_F1 | 70/30 | 12 | 55.31 | 52.3 | 5.442 |

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### EQUIVALENTS

The present disclosure provides, inter alia, fibers comprising recombinant spider silk polypeptides blended with cellulosic components. The present disclosure also provides methods of producing molded bodies and recombinant spider silk fibers blended with cellulosic components. While various specific embodiments have been illustrated and described, the above specification is not restrictive. It will be appreciated that various changes can be made without departing from the spirit and scope of the disclosure. Many variations will become apparent to those skilled in the art upon review of this specification.

1. A fiber comprising a blend of cellulose and recombinant spider silk polypeptide powder, wherein the fiber has a percentage by weight of cellulose ranging from 25%-90% and a percentage by weight of recombinant spider silk polypeptide powder ranging from 10%-75% and wherein the fiber has a tenacity of at least 10 cN/tex.

2. The fiber of claim 1, wherein the fiber has a tenacity of at least 20 cN/tex, wherein the fiber has a tenacity of at least 30 cN/tex, or wherein the fiber has a tenacity of at least 40 cN/tex.

3. (canceled)

4. (canceled)

5. The fiber of claim 1, wherein the tenacity is measured in accordance with ATSM D3822-14.

6. The fiber of claim 1, wherein the fiber has a percentage by weight of cellulose ranging from 50%-90% and a percentage by weight of recombinant spider silk polypeptide powder ranging from 10%-50%, wherein the fiber has a percentage by weight of cellulose ranging from 75%-90% and a percentage by weight of recombinant spider silk polypeptide powder ranging from 10%-25%, or wherein the fiber has a percentage by weight of cellulose ranging from 25%-90% and a percentage by weight of recombinant spider silk polypeptide powder ranging from 75%-10%.

7. (canceled)

8. (canceled)

9. The fiber of claim 1, wherein the fiber shrinks less than 5% by length when treated with water, wherein the fiber shrinks less than 4% by length when treated with water, or wherein the fiber shrinks less than 3% by length when treated with water.

10.-15. (canceled)

16. The method of claim 34, wherein the fiber has an improved luster after the fiber is treated by annealing the fiber with the alcohol.

17. The fiber of claim 1, wherein the recombinant silk polypeptide powder comprises a recombinant silk protein comprising SEQ ID NO: 1.

18. The fiber of claim 17, wherein the recombinant silk polypeptide powder has a purity of greater than 50%.

19. The fiber of claim 1, wherein the cellulose is microcrystalline cellulose, alpha cellulose, or dissolving cellulose.

20. (canceled)

21. (canceled)

22. The fiber of claim 1, wherein the fiber has a percentage elongation at break of at least 5%, a percentage elongation at break of at least 6%, or a percentage elongation at break of at least 7%.

23. (canceled)

24. (canceled)

25. The fiber of any of claim 22, wherein the percentage elongation at break is measured in accordance with ATSM D3822-14.

26. (canceled)

27. A staple yarn comprising a series of staple fibers formed from the fiber of claim 1.

28. A filament yarn comprising the fiber of claim 1 twisted around a common axis.

29. A method of producing a fiber comprising a blend of cellulose and recombinant spider silk polypeptide powder, the steps comprising:
a. dissolving cellulose and a recombinant spider silk polypeptide powder in a solvent to form a solution; and
b. extruding the solution to create a fiber, wherein the fiber has a percentage by weight of cellulose ranging from 25%-90% and a percentage by weight of recombinant spider silk polypeptide powder ranging from 10%-75% and wherein the fiber has a tenacity of at least 10 cN/tex.

30. The method of claim 29, wherein the solvent is NMMO monohydrate.

31. The method of claim 29, wherein the solution further comprises propyl gallate.

32. The method of claim 29, wherein extruding the fiber comprises extruding the fiber into a water bath.

33. The method of claim 29, wherein extruding the fiber comprises extruding the fiber into a water bath comprising an alcohol.

34. The method of claim 29, further comprising annealing the fiber with an alcohol.

35. The method of claim 29, wherein the fiber has a tenacity of at least 20 cN/tex, a tenacity of at least 30 cN/tex, or a tenacity of at least 40 cN/tex.

36. (canceled)

37. (canceled)

38. The method of claim 29, wherein the fiber has a percentage by weight of cellulose ranging from 50%-90% and a percentage by weight of recombinant spider silk polypeptide powder ranging from 10%-50%, wherein the fiber has a percentage by weight of cellulose ranging from 75%-90% and a percentage by weight of recombinant spider silk polypeptide powder ranging from 10%-25%, or wherein the fiber has a percentage by weight of cellulose ranging from 25%-90% and a percentage by weight of recombinant spider silk polypeptide powder ranging from 75%-10%.

39. (canceled)

40. The method of claim 29, further comprising extruding the fiber at an extrusion rate and winding the fiber on a first take-up device such that the jet stretch of the fiber is at least 7, at least 8, or at least 9.

41. (canceled)

42. (canceled)

43. The method of claim 29, wherein the fiber is transferred to one or more consecutive water baths.

44. The method of claim 43, wherein the fiber is not subject to any additional drawing as it is transferred between consecutive water baths.
